(19) United States
(12) Patent Application Publication
(10) Pub. No.: US 2024/0383853 A1
(43) Pub. Date: Nov. 21, 2024

(54) OXIMES AND THEIR USE IN TREATMENT OF GBA-RELATED DISEASES

(71) Applicant: ZEVRA DENMARK A/S, Copenhagen N (DK)

(72) Inventors: Søren NEVE, Lyngby (DK); William Dalby BROWN, Søborg (DK); Kenneth THIRSTRUP, Søborg (DK)

(21) Appl. No.: 18/695,892

(22) PCT Filed: Sep. 27, 2022
(86) PCT No.: PCT/IB2022/059203

§ 371 Date: Mar. 27, 2024

(30) Foreign Application Priority Data
Sep. 28, 2021 (EP) 21199458.7

(51) Int. Cl. C07D 211/14; A61K 31/4453; A61K 31/454; A61K 31/4545; C07D 211/34; C07D 213/54

(52) U.S. Cl. CPC C07D 211/14; A61K 31/4453; A61K 31/454; A61K 31/4545; C07D 211/34; C07D 213/54

(57) ABSTRACT

The present invention relates to oximes, their synthesis, and their use for increasing GBA activity and/or levels as well as treatment of GBA-related diseases, such as Parkinson's disease.

### TECHNICAL FIELD

The present invention relates to oximes, their synthesis, and their use for increasing GBA activity and/or levels as well as treatment of GBA-related diseases, such as Parkinson's disease.

### BACKGROUND

The lysosome functions as a crucial re-processing center in human cells, breaking down proteins and fatty substances, such as glycosphingolipids, into their basic building blocks that are then recycled. A set of rare genetic diseases, called lysosomal storage diseases (LSD), are the result of carrying a distinct mutation in both copies of certain genes which encode various lysosomal enzymes. Gaucher disease, the most common lysosomal storage disease, is the result of a mutation in both copies of the GBA1 gene that codes for the Glucocerebrosidase (GCase) enzyme. Such homozygous mutations in both copies of the GBA1 gene cause a severe loss of up to 95% of GCase activity. As a result of this critical loss of enzyme activity, the metabolism of certain glycosphingolipids is significantly impaired in Gaucher disease patients, leading to accumulation of Glucosylceramide (GluCer), the GCase enzyme's substrate. This accumulation leads to serious health issues and organ pathology.

Many of these GBA mutations are also found in patients with Parkinson's disease (PD). Heterozygous mutations as found in GBA mutation carriers (having one mutated GBA gene) are found to predispose for development of Parkinson's disease (Gan-Or et al., Neurology, 2015). Mutations in GBA are now considered one of the main genetic risk factors for Parkinson's disease. It has been estimated that at least 8% of patients with Parkinson's disease have mutations in the GBA gene, both mild and severe GBA mutations, including L444P heterozygotes. Also secondary deficiencies of GBA activity may be linked to Parkinson's disease.

State of the art compounds, Ambroxol and LTI-291 have been shown to increase GBA activity, an important effect in treatment of GBA-mediated disorders. In order to meet the medical need of treating GBA-mediated disorders, more and better compounds are needed.

### SUMMARY

The present inventors have developed a series of compounds that effectively act as GBA inducers with completely different structural chemotype compared to state of the art compounds Ambroxol and LTI-291. This renders the compounds of the present disclosure promising candidates for treatment of GBA-mediated disorders

In a first aspect, a compound of formula (I) is provided,

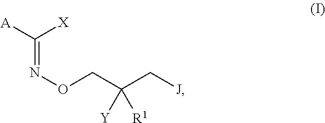

or a pharmaceutically acceptable salt thereof, wherein
R¹ is selected from the group consisting of: H, C₁₋₆ alkyl, and halogen;
Y is selected from the group consisting of: OH, and C₁₋₆ alkoxy;
X is selected from the group consisting of: chlorine and bromine;
J is an aliphatic cycle comprising at least one nitrogen atom, wherein the aliphatic cycle is optionally substituted; and wherein
A is selected from the group consisting of:

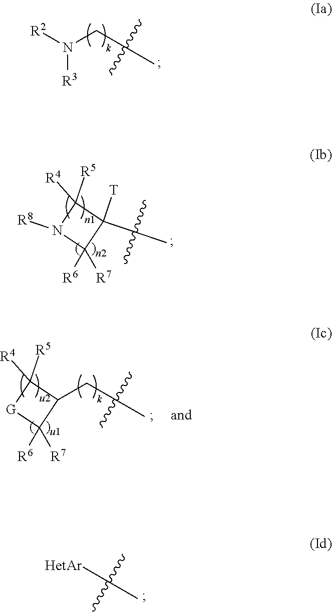

wherein each k is 1, 2, 3, or 4;
R² and R³ are independently of each other selected from the group consisting of: C₁₋₆ alkyl, C₁₋₆ acyl, and CO₂—C₁₋₆ alkyl;
n1, n2, u1, and u2 are independently of each other selected from the group consisting of: 1, 2, or 3;
each R⁴ is independently selected from the group consisting of: H, halogen, and C₁₋₄ alkyl, wherein each methylene group optionally is replaced by —O—;
each R⁵ is independently selected from the group consisting of: H, halogen, and C₁₋₄ alkyl, wherein each methylene group optionally is replaced by —O—;
each R⁶ is independently selected from the group consisting of: H, halogen, and C₁₋₄ alkyl, wherein each methylene group optionally is replaced by —O—;
each R⁷ is independently selected from the group consisting of: H, halogen, and C₁₋₄ alkyl, wherein each methylene group optionally is replaced by —O—;
R⁸ is selected from the group consisting of: H, and C₁₋₄ alkyl; G is selected from the group consisting of: —CH₂—, —CH(R⁹)—, —C(R⁹)₂—, —NH—, and —N(R⁹)—; wherein each R⁹ is independently selected from the group consisting of hydrogen, halogen, C₁₋₄ alkyl, wherein each methylene group optionally is replaced by —O—; and wherein HetAr is a heteroaryl optionally substituted by one or more R¹⁰, wherein each R¹⁰ is independently selected from the group consisting of: hydrogen, C₁₋₆ alkyl, halogen, hydroxy, C₁₋₆ alkoxy, amino, amido, and C₁₋₆ acyl;
and wherein T is selected from the group consisting of:

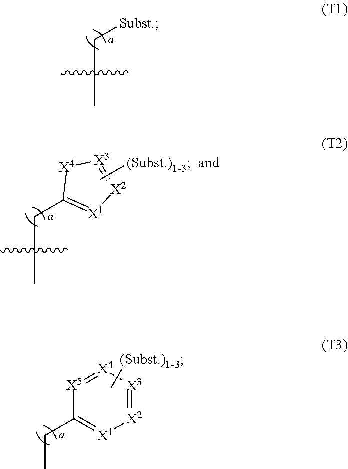

or any tautomer thereof,
wherein a is 0, 1, 2, or 3;
X¹, X², X³, X⁴, and X⁵ independently are selected from the group consisting of: C, CH, and N; and
each one, two, or three Subst. is independently selected from the group consisting of: hydrogen, alkyl, halogen, hydroxy, alkoxy, amino, amido, acyl, cycloalkyl, and heterocycloalkyl, wherein each methylene group of the alkyl is optionally replaced by —O—; with the proviso that when X is chlorine, A is not

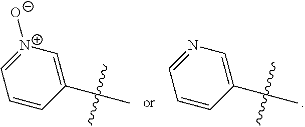 [SMILES: *c1ccc[n+]([O-])c1 ; *c1cccnc1]

In a second aspect, a pharmaceutical composition is provided comprising a compound as defined herein, and one or more pharmaceutically acceptable adjuvants, excipients, carriers, buffers and/or diluents.

In a third aspect, a method for treating a disease in a subject is provided comprising administering a compound as defined herein, wherein the disease is associated with reduced GBA levels and/or activity.

In a fourth aspect, a method for treating a disease in a subject is provided comprising administering a compound as defined herein, wherein the disease is associated with reduced GBA levels and/or activity.

In a fifth aspect, a method of increasing the GBA activity and/or levels is provided comprising contacting GBA with a compound as defined herein.

In a sixth aspect, a use of a compound as defined herein is provided for the manufacture of a medicament for the treatment of Parkinson's disease (PD).

### Definitions

With reference to substituents, the term “independently” refers to the situation where when more than one substituent is possible, the substituents may be the same or different from each other.

The potency, “EC_{1.5}” referred to herein is determined based on the dose response effects of the compounds as the concentration where “Percent GCase activity”=150% corresponding to at 1.5-fold induction of GCase activity.

As used herein, the term “pharmaceutically acceptable salt” refers to a salt used typically in the pharmaceutical field. Examples of the pharmaceutically acceptable salt include sodium salts, hydrochloride salts, magnesium salts, calcium salts, trifluoroacetic acid salts and potassium salts, but are not limited thereto. Further exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, malcate, gentisinate, fumarate, gluconate, glucuronate, saccharate, formate, benzoate, glutamate, methanesulfonate, ethanesulfonate, benzenesulfonate, p-toluenesulfonate, and pamoate.

The term “alkyl” refers to a straight or branched hydrocarbon chain radical consisting of carbon and hydrogen atoms, and may be straight or branched, substituted or unsubstituted. In some preferred embodiments, the alkyl group may consist of 1 to 12 carbon atoms, e.g. 1 carbon atom, 2 carbon atoms, 3 carbon atoms, 4 carbon atoms etc., up to and including 12 carbon atoms. Exemplary alkyl groups include, but are not limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (iso-propyl), n-butyl, n-pentyl, 1,1-dimethylethyl (t-butyl) and 3-methylhexyl. Unless stated otherwise specifically in the specification, an alkyl group is optionally substituted by one or more of any suitable substituents. An alkyl group can be mono-, di-, tri- or tetra-valent, as appropriate to satisfy valence requirements.

The term “aliphatic cycle”, as used herein, means a hydrocarbon cycle that is completely saturated or that contains one or more units of unsaturation but is non-aromatic. Unless otherwise specified, aliphatic cycles contain 1-20 aliphatic carbon atoms, In some embodiments, aliphatic cycles contain 1-10 aliphatic carbon atoms. In other embodiments, aliphatic cycles contain 1-8 aliphatic carbon atoms. In still other embodiments, aliphatic cycles contain 1-6 aliphatic carbon atoms, and in yet other embodiments aliphatic cycles contain 1-4 aliphatic carbon atoms.

Generally, suitable substituents for substituted groups disclosed herein independently include, but are not limited to, alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a}, —N(R^{a})S(O)₂R^{a}, —S(O)OR^{a}, —S(O)₂OR^{a}, —S(O)N(R^{a})₂, —S(O)₂N(R^{a})₂, or PO₃(R^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl.

The term “cycloalkyl” refers to a monocyclic or polycyclic radical that contains carbon and hydrogen, and may be saturated, or partially unsaturated. In some preferred embodiments, cycloalkyl groups include groups having from 3 to 12 ring atoms (i.e. (C₃₋₁₂)cycloalkyl or C(₃₋₁₂)cycloalkyl). Whenever it appears herein, a numerical range such as “3 to 12” in (C₃₋₁₂)cycloalkyl or C(₃₋₁₂)cycloalkyl refers to each integer in the given range—e.g., “3 to 12 carbon atoms” means that the cycloalkyl group may consist of 3 carbon atoms, 4 carbon atoms, 5 carbon atoms, etc., up to and including 12 carbon atoms. Illustrative examples of cycloalkyl groups include, but are not limited to cyclopropyl, cyclobutyl, cyclopentyl, cyclopentenyl, cyclohexyl, cyclohexenyl, cycloseptyl, cyclooctyl, cyclononyl, cyclodecyl, norbornyl, and the like.

The term “alkoxy” refers to the group —O-alkyl. In some preferred embodiments, the alkoxy group contains from 1 to 12 carbon atoms of a straight, branched, cyclic configuration and combinations thereof attached to the parent structure through an oxygen. Examples of alkoxy include, but are not limited to, methoxy, ethoxy, propoxy, isopropoxy, cyclopropyloxy and cyclohexyloxy.

The term “acyl” refers to R^{c}—(C═O)— wherein R^{c} include, but is not limited to, alkyl, heteroalkyl, alkenyl, alkynyl, cycloalkyl, heterocycloalkyl, aryl, aralkyl, heteroaryl. The acyl is attached to the parent structure through the carbonyl functionality.

The term “amino” or “amine” refers to a —N(R^{a})₂ radical group, where each R^{a} is independently hydrogen, alkyl, (halo)alkyl, alkenyl, alkynyl, carbocyclyl, carbocyclylalkyl, aryl, aralkyl, heterocycloalkyl, heterocycloalkylalkyl, heteroaryl or heteroarylalkyl, unless stated otherwise. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl, 1-piperazinyl, and 4-morpholinyl.

The term “amide” or “amido” refers to a chemical moiety with formula —(C═O)N(R^{d})₂ or —NH(C═O)R^{d}, where R^{d} is selected from the group consisting of hydrogen, alkyl, alkenyl, alkynyl, carbocyclyl, carbocyclylalkyl, cycloalkyl, aryl, and heteroaryl. The R^{d} of —N(R^{d})₂ of the amide may optionally be taken together with the nitrogen to which it is attached to form a 4-, 5-, 6- or 7-membered ring. Unless stated otherwise specifically in the specification, an amide group is optionally substituted independently by one or more of the substituents as described herein as suitable substitution groups.

The term “haloalkyl” refers to an alkyl radical, as defined above, that is substituted by one or more halogen atoms. The term “alkyl” thus includes “haloalkyl”. Examples of haloalkyl include, but are not limited to, trifluoromethyl, difluoromethyl, 2,2,2-trifluoroethyl, 1-fluoromethyl-2-fluoroethyl, and the like.

The term “halo”, “halide”, or, alternatively, “halogen” is intended to mean fluoro, chloro, bromo or iodo.

The term “aromatic” means an unsaturated, cyclic and planar hydrocarbon group with a delocalized conjugated π system having 4n+2 π electrons, where n is an integer having a value of 0, 1, 2, 3, and so on. In some embodiments, the aromatic group is an “aryl” (abbreviated as Ar), which refers to an aromatic radical with six to ten ring atoms (e.g., (C₆₋₁₀)aromatic or (C₆₋₁₀)aryl) which has at least one ring having a conjugated pi electron system which is carbocyclic (e.g., phenyl, fluorenyl, and naphthyl).

The term “aralkyl” or “arylalkyl” refers to an (aryl)alkyl-radical where aryl and alkyl are as disclosed herein.

The term “heteroaryl” or “heteroaromatic refers to a 5- to 18-membered aromatic radical (e.g., (C₅₋₁₃)heteroaryl) that includes one or more ring heteroatoms selected from nitrogen, oxygen and sulfur, and which may be a monocyclic, bicyclic, tricyclic or tetracyclic ring system. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl).

The term “tautomer” relate to structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond.

The symbol “ [symbol] ”, displayed perpendicular to a bond, indicates the point at which the displayed moiety is attached to the remainder of the molecule.

### Compounds

In one embodiment, a compound of formula (I) is provided,

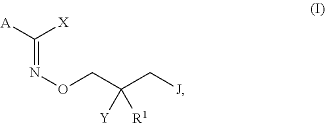

or a pharmaceutically acceptable salt thereof, wherein
R¹ is selected from the group consisting of: H, C₁₋₆ alkyl, and halogen;
Y is selected from the group consisting of: OH, and C₁₋₆ alkoxy;
X is selected from the group consisting of: chlorine and bromine;
J is an aliphatic cycle comprising at least one nitrogen atom, wherein the aliphatic cycle is optionally substituted; and wherein
A is selected from the group consisting of:

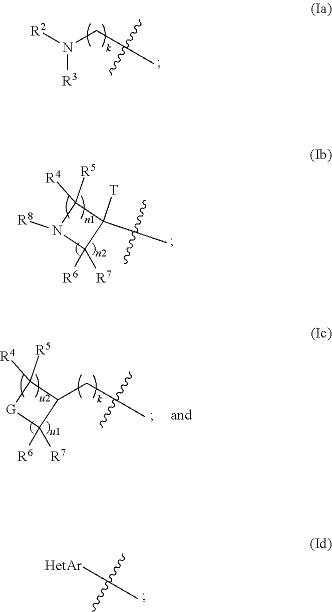

wherein each k is 1, 2, 3, or 4;
R² and R³ are independently of each other selected from the group consisting of: C₁₋₆ alkyl, C₁₋₆ acyl, and CO₂—C₁₋₆ alkyl;
n1, n2, u1, and u2 are independently of each other selected from the group consisting of: 1, 2, or 3;
each R⁴ is independently selected from the group consisting of: H, halogen, and C₁₋₄ alkyl, wherein each methylene group optionally is replaced by —O—;
each R⁵ is independently selected from the group consisting of: H, halogen, and C₁₋₄ alkyl, wherein each methylene group optionally is replaced by —O—;
each R⁶ is independently selected from the group consisting of: H, halogen, and C₁₋₄ alkyl, wherein each methylene group optionally is replaced by —O—;
each R⁷ is independently selected from the group consisting of: H, halogen, and C₁₋₄ alkyl, wherein each methylene group optionally is replaced by —O—;
R⁸ is selected from the group consisting of: H, and C₁₋₄ alkyl;
G is selected from the group consisting of: —CH₂—, —CH(R⁹)—, —C(R⁹)₂—, —NH—, and —N(R⁹)—; wherein each R⁹ is independently selected from the group consisting of hydrogen, halogen, C₁₋₄ alkyl, wherein each methylene group optionally is replaced by —O—; and wherein HetAr is a heteroaryl optionally substituted by one or more R¹⁰, wherein each R¹⁰ is independently selected from the group consisting of: hydrogen, C₁₋₆ alkyl, halogen, hydroxy, C₁₋₆ alkoxy, amino, amido, and C₁₋₆ acyl;
and wherein T is selected from the group consisting of:

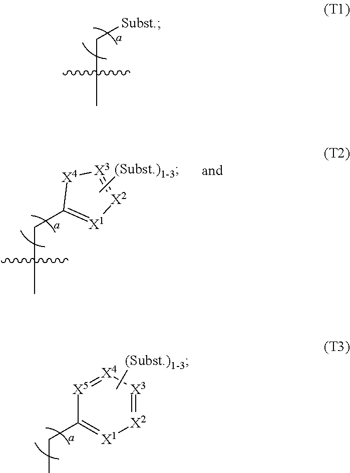

or any tautomer thereof,
wherein a is 0, 1, 2, or 3;
X¹, X², X³, X⁴, and X⁵ independently are selected from the group consisting of: C, CH, and N; and
each one, two, or three Subst. is independently selected from the group consisting of: hydrogen, alkyl, halogen, hydroxy, alkoxy, amino, amido, acyl, cycloalkyl, and heterocycloalkyl, wherein each methylene group of the alkyl is optionally replaced by —O—;
with the proviso that when X is chlorine, A is not

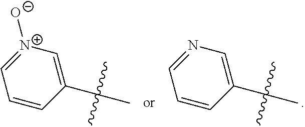 [SMILES: *c1ccc[n+]([O-])c1 ; *c1cccnc1]

In one embodiment, the compound as defined herein is provided, wherein J is of formula (J1):

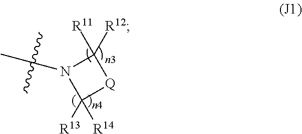

wherein n3 and n4 are independently 1, 2, or 3;
each R¹¹ is independently selected from the group consisting of: H, halogen, and C₁₋₄ alkyl, wherein each methylene group optionally is replaced by —O—;
each R¹² is independently selected from the group consisting of: H, halogen, and C₁₋₄ alkyl, wherein each methylene group optionally is replaced by —O—;
each R¹³ is independently selected from the group consisting of: H, halogen, and C₁₋₄ alkyl, wherein each methylene group optionally is replaced by —O—;
each R¹⁴ is independently selected from the group consisting of: H, halogen, and C₁₋₄ alkyl, wherein each methylene group optionally is replaced by —O—;
Q is selected from the group consisting of: a bond, —CH₂—, —CH(R¹⁵)—, —C(R¹⁵)₂—, —NH—, and —N(R¹⁵)—; and wherein each R¹⁵ is independently selected from the group consisting of hydrogen, halogen, C₁₋₄ alkyl, wherein each methylene group optionally is replaced by —O—; and wherein a pair of R¹¹ and R¹³ are optionally linked together to form a ring.

In one embodiment, the compound is provided wherein Q is selected from the group consisting of: a bond, —CH₂—, —CHF—, —N(Me)-, and —NH—. In one embodiment, R¹¹, R¹², R¹³, and R¹⁴ are all hydrogen.

In one embodiment, n3 and n4 are each 2.

In one embodiment, the compound is provided wherein J is selected from the group consisting of:

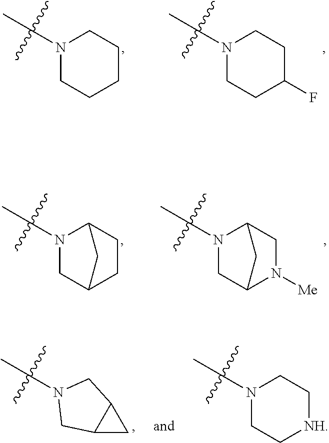 [SMILES: *N1CCCCC1 ; *N1CCC(F)CC1 ; *N1CC2CCC1C2 ; *N1CC2CC1CN2C ; *N1CC2CC2C1 ; *N1CCNCC1]

In one embodiment, the compound is provided wherein Y is OH.

In one embodiment, the compound is provided wherein R¹ is hydrogen or methyl.

In one embodiment, the compound is provided wherein A is of formula (Ia);

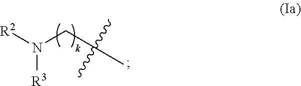

k is 2, R³ is C₁₋₃ alkyl, and R² is C₁₋₃ alkyl or CO₂tBu.

In one embodiment, the compound is provided wherein A is of formula (Ib);

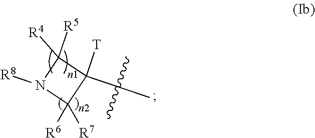

wherein n1 and n2 are each 2; R⁴, R⁵, R⁶, and R⁷ are each hydrogen, R⁸ is hydrogen or C₁₋₃ alkyl; and T is of formula (T3);

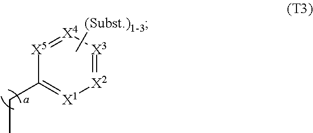

wherein a is 0 or 1; wherein X¹, X², and/or X³ is N and the remainder of X¹—X⁵ are independently C or CH; and wherein each one, two, or three Subst. is independently selected from the group consisting of: hydrogen, C₁₋₄ alkyl, halogen, hydroxy, C₁₋₄ alkoxy, and C₁₋₄ acyl.

In one embodiment, only one Subst. is present and is methyl. In one embodiment, only one Subst. is present and is chlorine. In one embodiment, all Subst. are each hydrogen.

In one embodiment, the compound is provided wherein A is of formula (Ib);

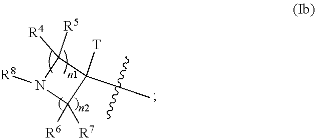

wherein n1 and n2 are each 2; R⁴, R⁵, R⁶, and R⁷ are each hydrogen; R⁸ is hydrogen or C₁₋₃ alkyl; and T is of formula (T1);

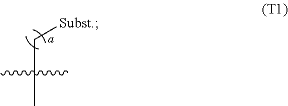

wherein a is 0 and Subst is C₁₋₆ alkyl, wherein each methylene group is optionally replaced by —O—.

In one embodiment, the compound is provided wherein A is of formula (Ic);

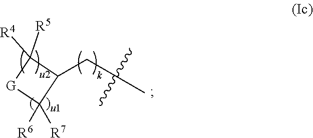

wherein k is 1, 2, or 3; u1 and u2 are each 1 or 2; R⁴, R⁵, R⁶, and R⁷ are each hydrogen; and G is selected from the group consisting of: a bond, —CH₂—, —NH—, and —N(C₁₋₃ alkyl)-.

In one embodiment, the compound is provided wherein A is of formula (Id);

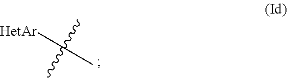

wherein HetAr is a C₅₋₁₃ heteroaryl comprising one or more ring heteroatoms selected from nitrogen, oxygen and sulfur, and which is monocyclic, bicyclic, or tricyclic.

In one embodiment, the compound is provided wherein A is selected from the group consisting of:

In one embodiment, the compound is selected from the group consisting of:

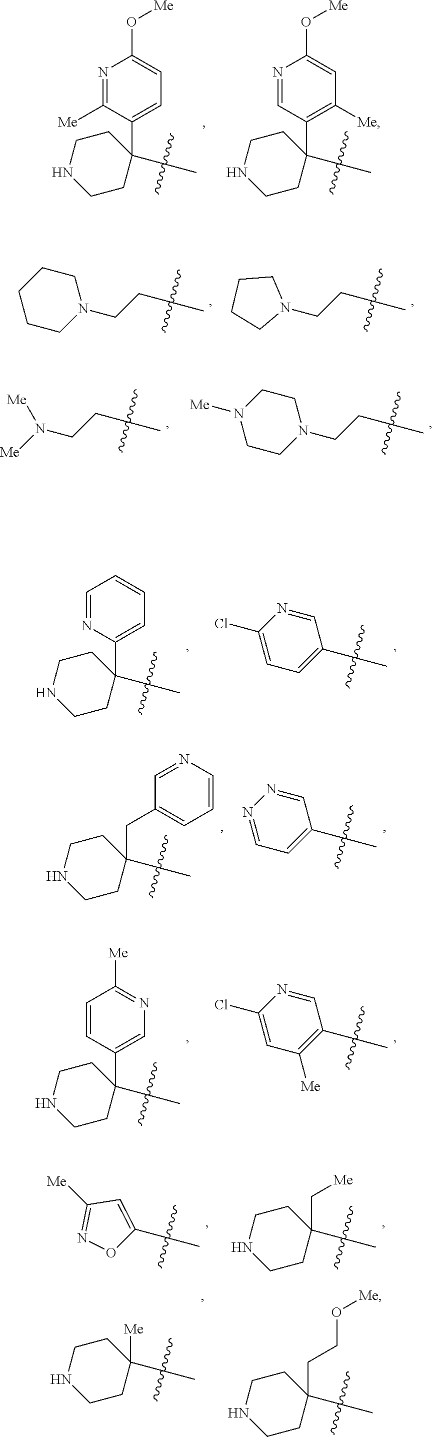

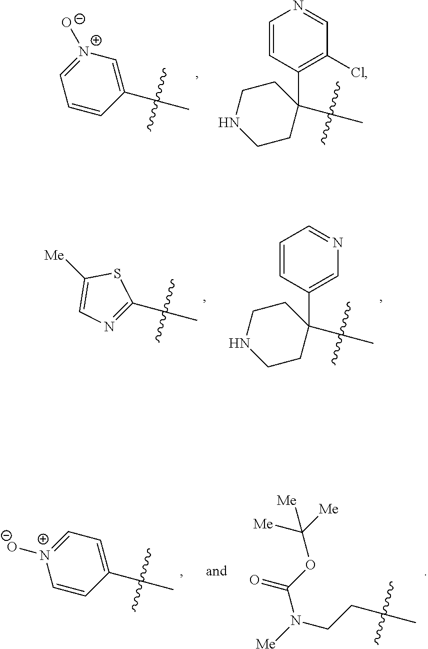 [SMILES: *c1ccc[n+]([O-])c1 ; *C1(c2ccncc2Cl)CCNCC1 ; *c1ncc(C)s1 ; *C1(c2cccnc2)CCNCC1 ; *c1cc[n+]([O-])cc1 ; *CCN(C)C(=O)OC(C)(C)C]

In one embodiment, the compound is selected from the group consisting of:

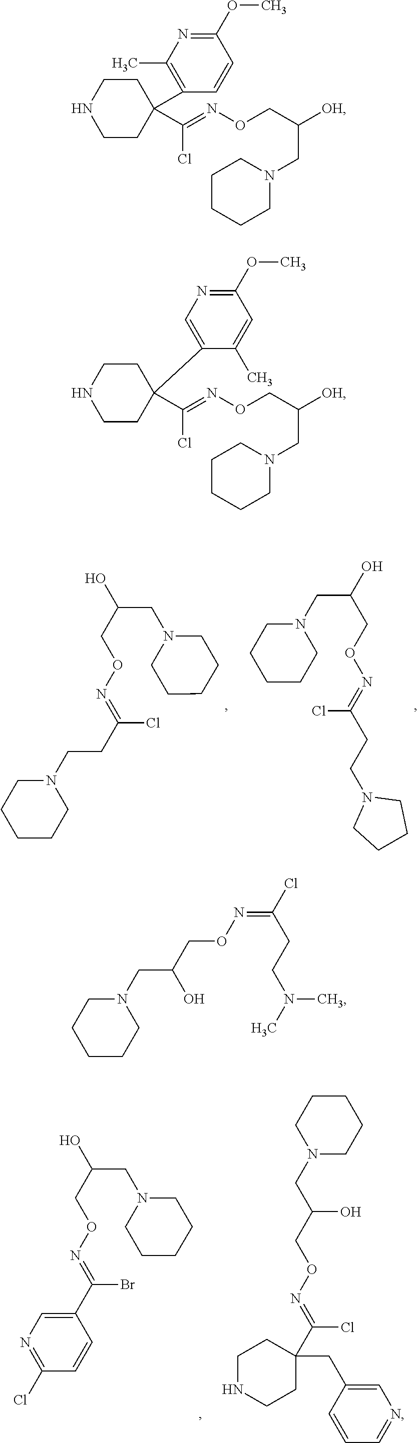

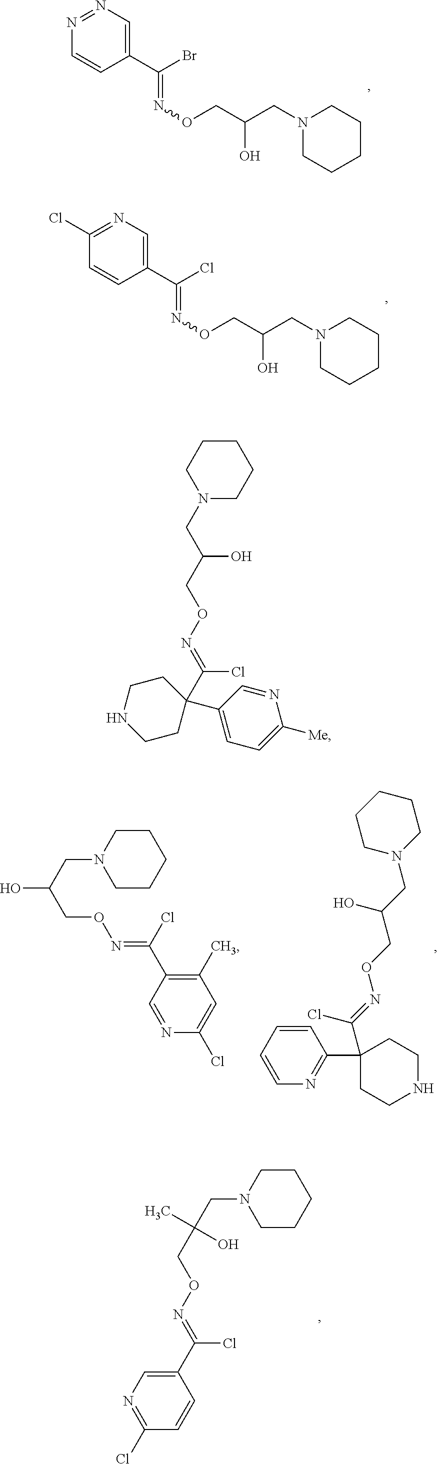

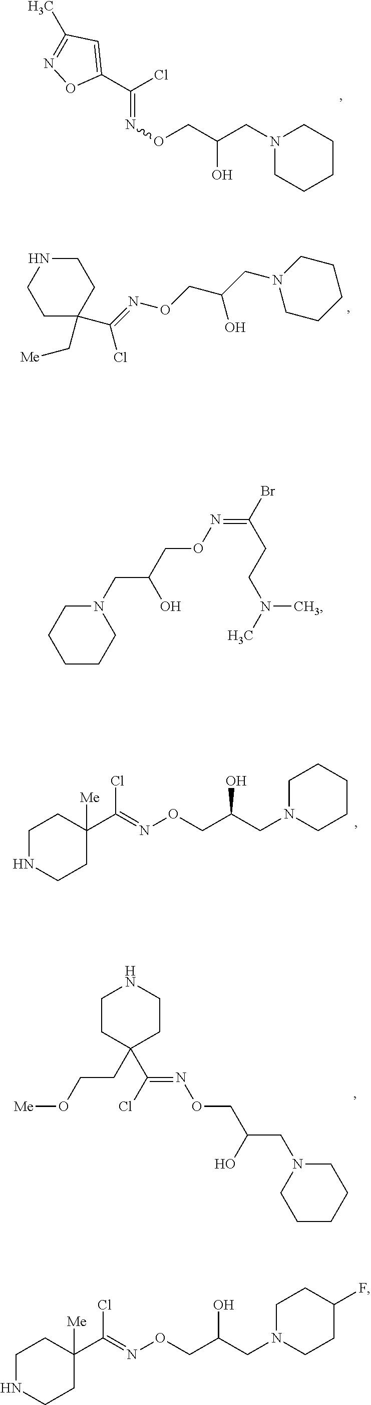 [SMILES: Cc1cc(C(Cl)=NOCC(O)CN2CCCCC2)on1 ; CCC1(/C(Cl)=N/OCC(O)CN2CCCCC2)CCNCC1 ; CN(C)CC/C(Br)=N\OCC(O)CN1CCCCC1 ; CC1(/C(Cl)=N/OC[C@@H](O)CN2CCCCC2)CCNCC1 ; COCCC1(/C(Cl)=N/OCC(O)CN2CCCCC2)CCNCC1 ; CC1(/C(Cl)=N/OCC(O)CN2CCC(F)CC2)CCNCC1]

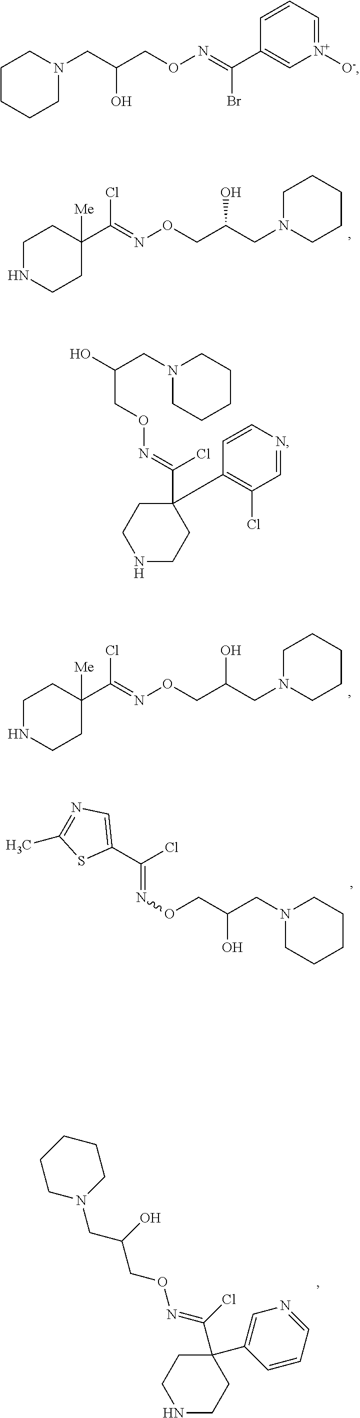

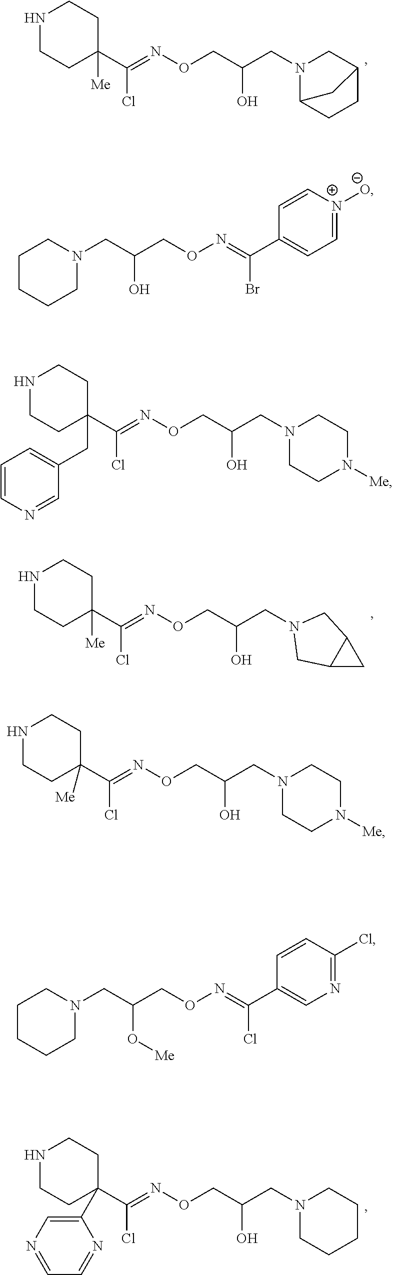

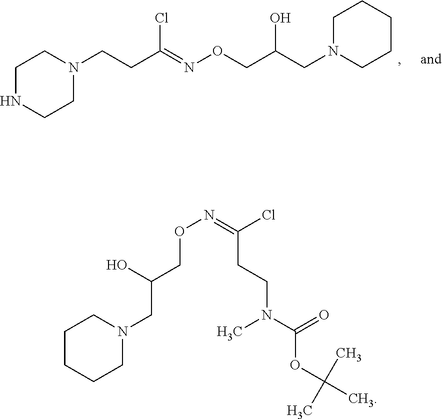 [SMILES: OC(CO/N=C(\Cl)CCN1CCNCC1)CN1CCCCC1 ; CN(CC/C(Cl)=N\OCC(O)CN1CCCCC1)C(=O)OC(C)(C)C]

### GBA Inducers

The compounds of the present disclosure are capable of inducing glucocerebrosidase (GBA) enzyme activity and/or GBA levels. Hence, the compounds of the present disclosure are GBA inducers, i.e. capable of inducing increased GBA enzyme levels and/or activity. In one embodiment, the compound provided is a GBA inducer.

In one embodiment, the compound is provided for use in a method of increasing GBA levels and/or activity. This effect can be readily determined using the assay provided in Example 2.

In one embodiment, the compound is provided which is capable of increasing said GBA activity at least 1.5-fold, such as at least 2-fold, for example at least 2.5-fold, such as at least 3-fold. In one embodiment, the method provides for increasing GBA activity at least 1.5-fold, such as at least 2-fold, for example at least 2.5-fold, such as at least 3-fold.

In one embodiment, the GBA activity is increased to 50% or more of hypothetical wild-type levels, such as 50-60%, such as 60-70%, such as 70-80%, such as 80-90%, such as 90-100%, such as 100-110%, such as 110-120%, such as 120-130%, such as 130-140%, such as 140-150% of hypothetical wild-type levels.

In one embodiment, the EC_{1.5} of the compound is 150 μM or less, such as 140 μM or less, such as 130 μM or less, such as 120 μM or less, such as 110 μM or less, such as 100 μM or less, such as 90 μM or less, such as 80 μM or less, such as 70 μM or less, such as 60 μM or less, preferably wherein the EC_{1.5} is 50 μM or less, such as 40 μM or less, such as 30 μM or less, such as 20 μM or less, such as 10 μM or less.

In one embodiment, the Emax % of the compound is 80% or more, such as 100% or more, such as 120% or more, such as 140% or more, such as 160% or more, such as 180% or more, such as 200% or more, such as 220% or more, such as 240% or more, such as 260% or more, such as 280% or more, such as 300% or more.

### Pharmaceutical Compositions

In one embodiment, a pharmaceutical composition is provided comprising a compound as defined herein, and one or more pharmaceutically acceptable adjuvants, excipients, carriers, buffers and/or diluents.

### Therapy

The compounds of the present disclosure are important for use in therapy. In one embodiment, a method for treating a disease in a subject comprising administering a compound as defined herein is provided, wherein the disease is associated with reduced GBA levels and/or activity.

In one embodiment, the method is provided wherein the disease treated is Parkinson's disease (PD). In one embodiment, a compound as defined herein is provided for use in the treatment of Parkinson's disease.

In one embodiment, use of a compound as defined herein is provided for the manufacture of a medicament for the treatment of Parkinson's disease (PD).

### Items

1. A compound of formula (I),

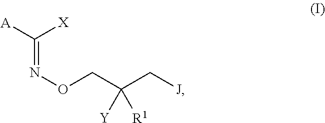

or a pharmaceutically acceptable salt thereof, wherein
R¹ is selected from the group consisting of: H, C₁₋₆ alkyl, and halogen;
Y is selected from the group consisting of: OH, and C₁₋₆ alkoxy;
X is selected from the group consisting of: chlorine and bromine;
J is an aliphatic cycle comprising at least one nitrogen atom, wherein the aliphatic cycle is optionally substituted; and wherein
A is selected from the group consisting of:

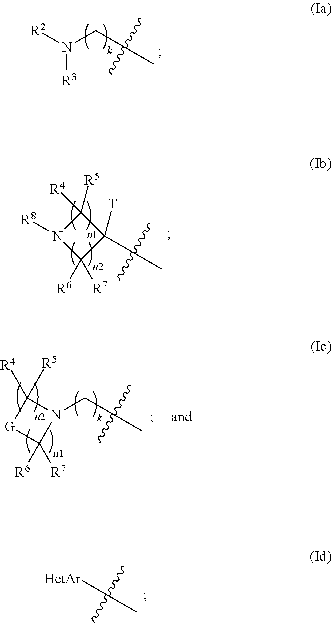

wherein each k is 1, 2, 3, or 4; R² and R³ are independently of each other selected from the group consisting of: C₁₋₆ alkyl, C₁₋₆ acyl, and CO₂—C₁0.6 alkyl;
n1, n2, u1, and u2 are independently of each other selected from the group consisting of: 1, 2, or 3;
each R⁴ is independently selected from the group consisting of: H, halogen, and C₁₋₄ alkyl, wherein each methylene group optionally is replaced by —O—;
each R⁵ is independently selected from the group consisting of: H, halogen, and C₁₋₄ alkyl, wherein each methylene group optionally is replaced by —O—;
each R⁶ is independently selected from the group consisting of: H, halogen, and C₁₋₄ alkyl, wherein each methylene group optionally is replaced by —O—;
each R⁷ is independently selected from the group consisting of: H, halogen, and C₁₋₄ alkyl, wherein each methylene group optionally is replaced by —O—;
R⁸ is selected from the group consisting of: H, and C₁₋₄ alkyl;
G is selected from the group consisting of: —CH₂—, —CH(R⁹)—, —C(R⁹)₂—, —NH—, and —N(R⁹)—;
wherein each R⁹ is independently selected from the group consisting of hydrogen, halogen, C₁₋₄ alkyl, wherein each methylene group optionally is replaced by —O—; and wherein
HetAr is a heteroaryl optionally substituted by one or more R¹⁰, wherein each R¹⁰ is independently selected from the group consisting of: hydrogen, C₁₋₆ alkyl, halogen, hydroxy, C₁₋₆ alkoxy, amino, amido, and C₁₋₆ acyl;
and wherein T is selected from the group consisting of:

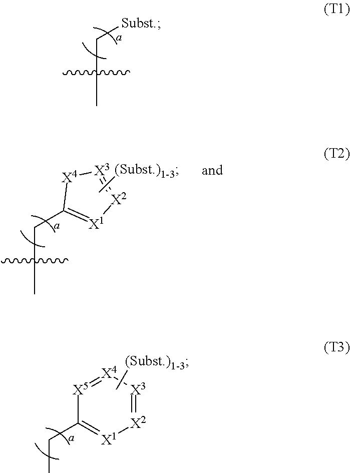

or any tautomer thereof,
wherein a is 0, 1, 2, or 3;
X¹, X², X³, X⁴, and X⁵ independently are selected from the group consisting of: C, CH, and N; and
each one, two, or three Subst. is independently selected from the group consisting of: hydrogen, alkyl, halogen, hydroxy, alkoxy, amino, amido, acyl, cycloalkyl, and heterocycloalkyl, wherein each methylene group of the alkyl is optionally replaced by —O—;
with the proviso that when X is chlorine, A is not

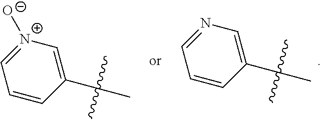 [SMILES: *c1ccc[n+]([O-])c1 ; *c1cccnc1]

2. The compound according to any one of the preceding items, wherein J is of formula (J1):

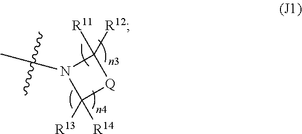

wherein n3 and n4 are independently 1, 2, or 3;
each R¹¹ is independently selected from the group consisting of: H, halogen, and C₁₋₄ alkyl, wherein each methylene group optionally is replaced by —O—;
each R¹² is independently selected from the group consisting of: H, halogen, and C₁₋₄ alkyl, wherein each methylene group optionally is replaced by —O—;
each R¹³ is independently selected from the group consisting of: H, halogen, and C₁₋₄ alkyl, wherein each methylene group optionally is replaced by —O—;
each R¹⁴ is independently selected from the group consisting of: H, halogen, and C₁₋₄ alkyl, wherein each methylene group optionally is replaced by —O—;
Q is selected from the group consisting of: a bond, —CH₂—, —CH(R¹⁵)—, —C(R¹⁵)₂—, —NH—, and —N(R¹⁵)—; and wherein each R¹⁵ is independently selected from the group consisting of hydrogen, halogen, C₁₋₄ alkyl, wherein each methylene group optionally is replaced by —O—; and wherein a pair of R¹¹ and R¹³ are optionally linked together to form a ring.
3. The compound according to any one of the preceding items, wherein Q is selected from the group consisting of: a bond, —CH₂—, —CHF—, —N(Me)-, and —NH—.
4. The compound according to any one of the preceding items, wherein R¹¹, R¹², R¹³, and R¹⁴ are all hydrogen.
5. The compound according to any one of the preceding items, wherein n3 and n4 are each 2.
6. The compound according to any one of the preceding items, wherein J is selected from the group consisting of:

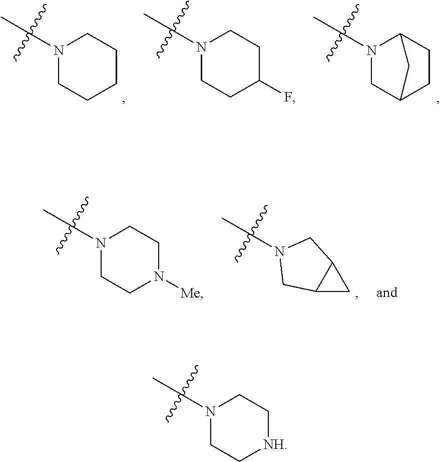 [SMILES: *N1CCCCC1 ; *N1CCC(F)CC1 ; *N1CC2CCC1C2 ; *N1CCN(C)CC1 ; *N1CC2CC2C1 ; *N1CCNCC1]

7. The compound according to any one of the preceding items, wherein Y is OH.
8. The compound according to any one of the preceding items, wherein R¹ is hydrogen or methyl.
9. The compound according to any one of the preceding items, wherein A is of formula (Ia);

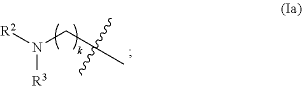

k is 2, R³ is C₁₋₃ alkyl, and R² is C₁₋₃ alkyl or CO₂tBu.
10. The compound according to any one of the preceding items, wherein A is of formula (Ib);

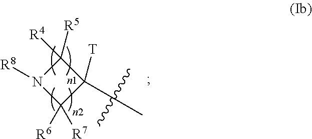

wherein n1 and n2 are each 2; R⁴, R⁵, R⁶, and R⁷ are each hydrogen, R⁸ is hydrogen or C₁₋₃ alkyl; and T is of formula (T3);

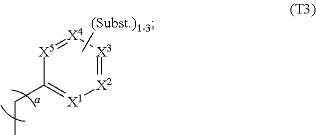

wherein a is 0 or 1; wherein X¹, X², and/or X³ is N and the remainder of X¹—X⁵ are independently C or CH; and wherein each one, two, or three Subst. is independently selected from the group consisting of: hydrogen, C₁₋₄ alkyl, halogen, hydroxy, C₁₋₄ alkoxy, and C₁₋₄ acyl.
11. The compound according to any one of the preceding items, wherein only one Subst. is present and is methyl.
12. The compound according to any one of the preceding items, wherein only one Subst. is present and is chlorine.
13. The compound according to any one of the preceding items, wherein all Subst. are each hydrogen.
14. The compound according to any one of the preceding items, wherein A is of formula (Ib);

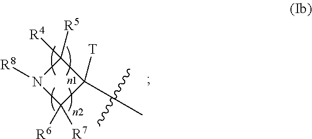

wherein n1 and n2 are each 2; R⁴, R⁵, R⁶, and R⁷ are each hydrogen; R⁸ is hydrogen or C₁₋₃ alkyl; and T is of formula (T1);

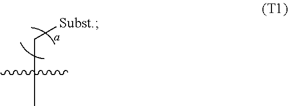

wherein a is 0 and Subst is C₁₋₆ alkyl, wherein each methylene group is optionally replaced by —O—.
15. The compound according to any one of the preceding items, wherein A is of formula (Ic);

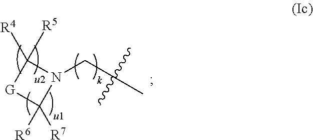

wherein k is 1, 2, or 3; u1 and u2 are each 1 or 2; R⁴, R⁵, R⁶, and R⁷ are each hydrogen; and G is selected from the group consisting of: a bond, —CH₂—, —NH—, and —N(C₁₋₃ alkyl)-.
16. The compound according to any one of the preceding items, wherein A is of formula (Id);

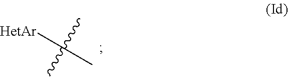

wherein HetAr is a C₅₋₁₃ heteroaryl comprising one or more ring heteroatoms selected from nitrogen, oxygen and sulfur, and which is monocyclic, bicyclic, or tricyclic.
17. The compound according to any one of the preceding items, wherein A is selected from the group consisting of:

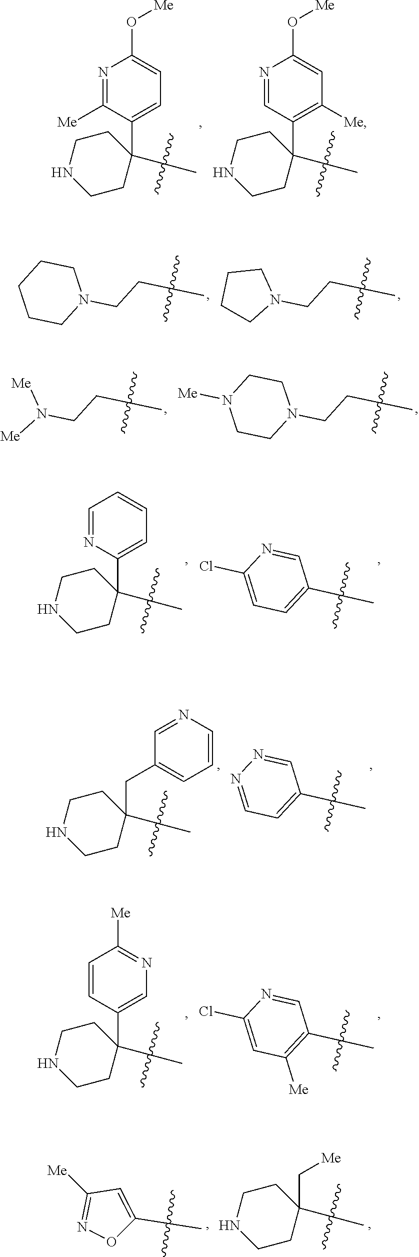

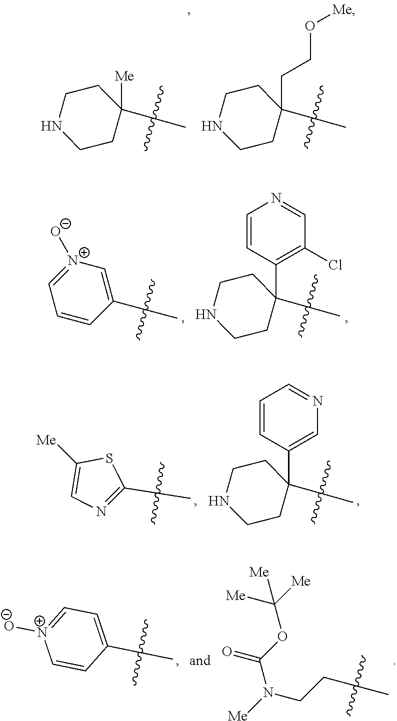

18. The compound according to any one of the preceding items, wherein the compound is selected from the group consisting of:

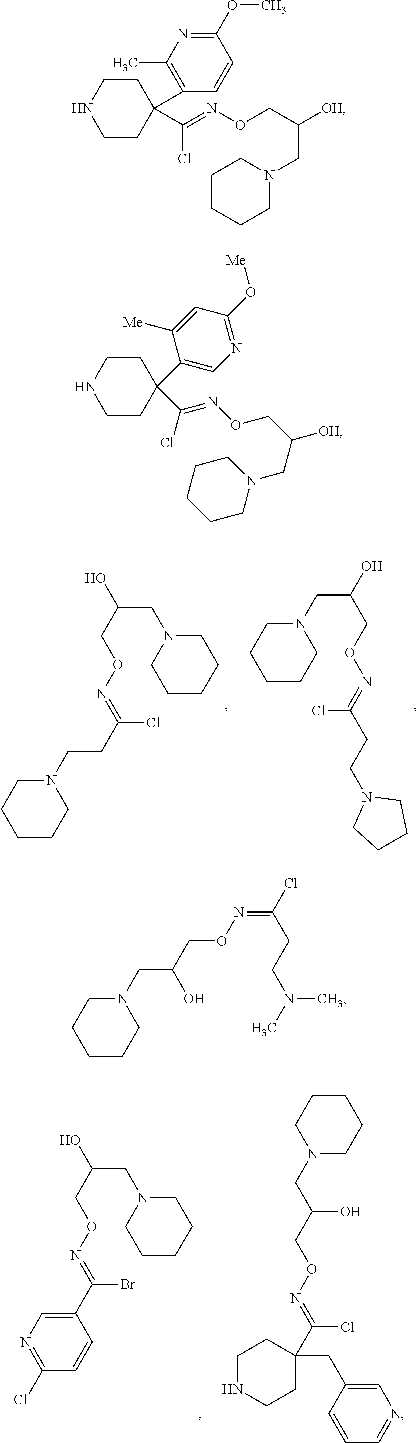

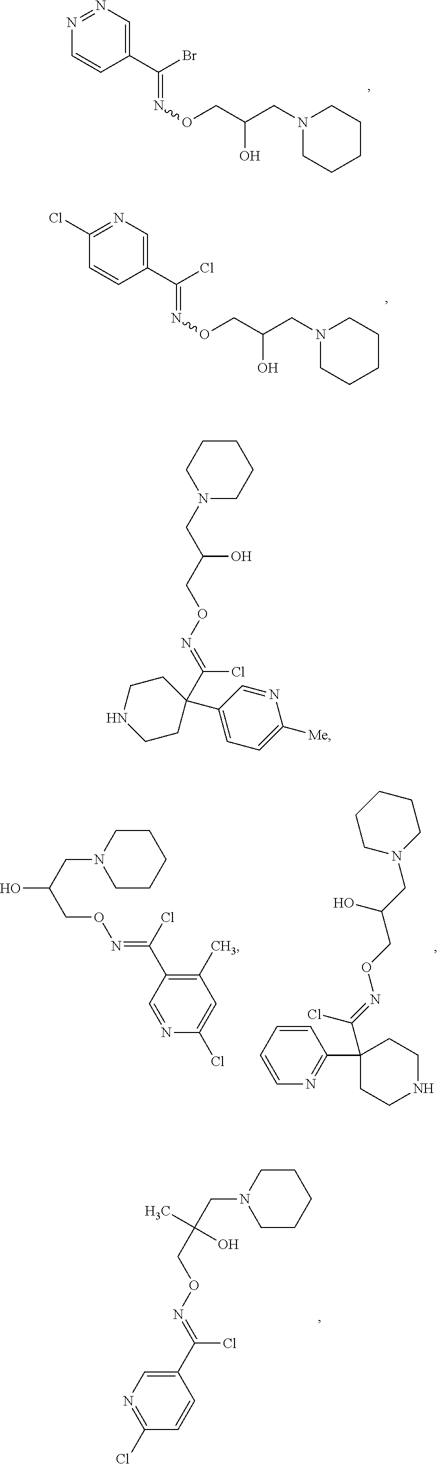

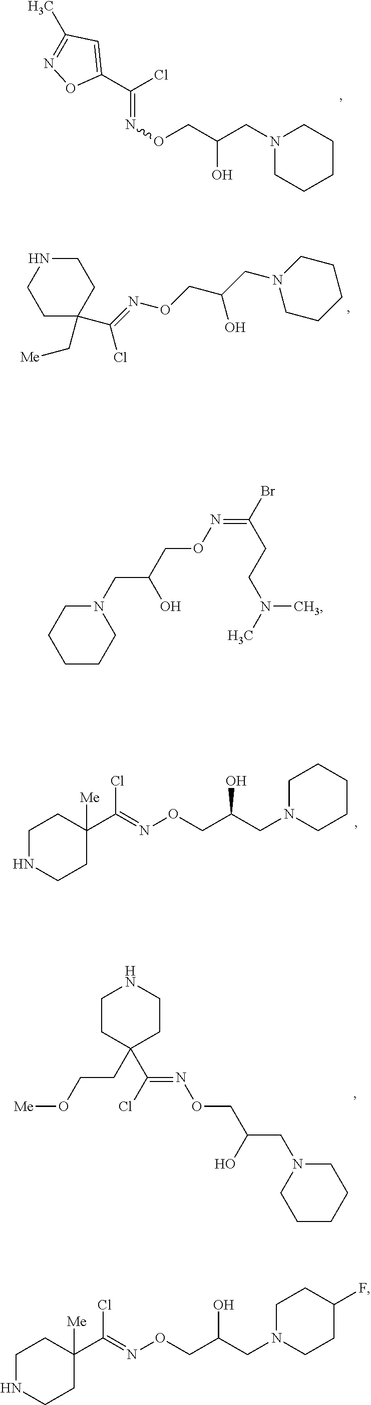 [SMILES: Cc1cc(C(Cl)=NOCC(O)CN2CCCCC2)on1 ; CCC1(/C(Cl)=N/OCC(O)CN2CCCCC2)CCNCC1 ; CN(C)CC/C(Br)=N\OCC(O)CN1CCCCC1 ; CC1(/C(Cl)=N/OC[C@@H](O)CN2CCCCC2)CCNCC1 ; COCCC1(/C(Cl)=N/OCC(O)CN2CCCCC2)CCNCC1 ; CC1(/C(Cl)=N/OCC(O)CN2CCC(F)CC2)CCNCC1]

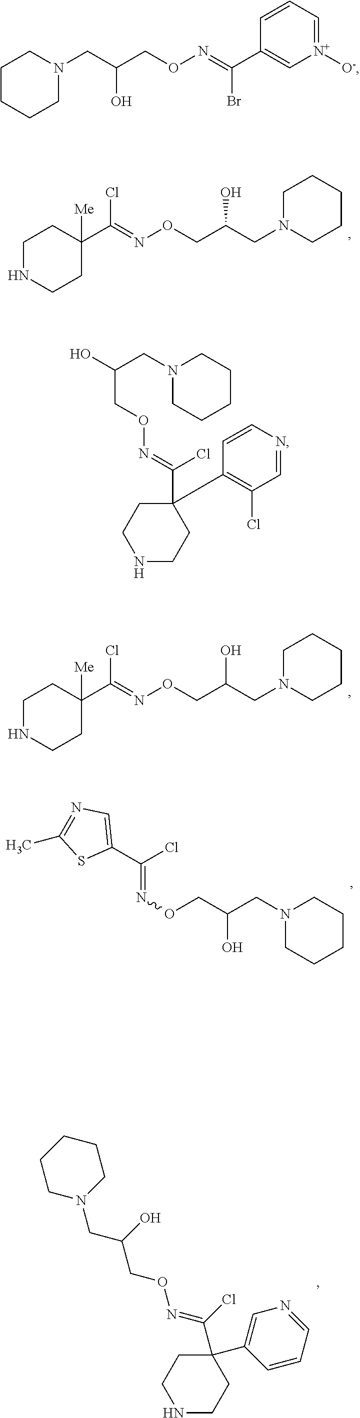

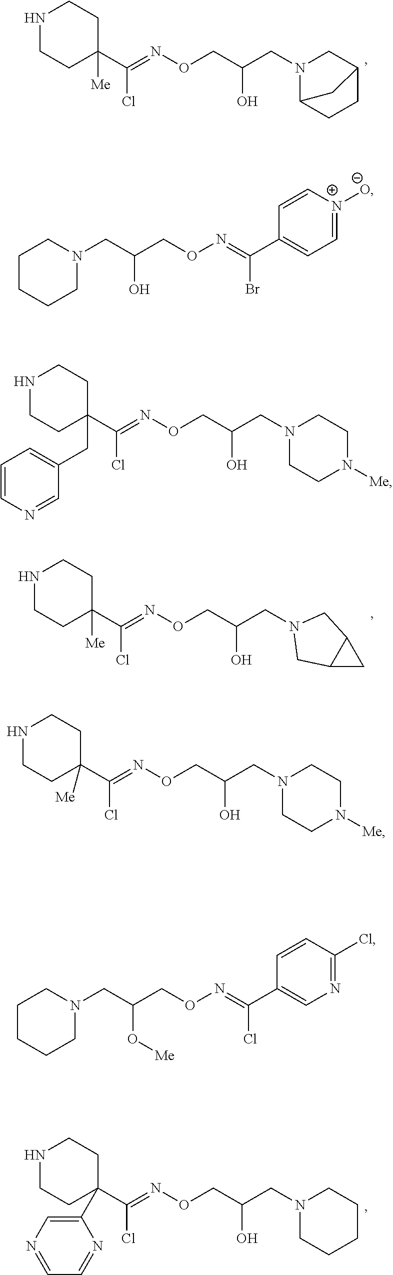

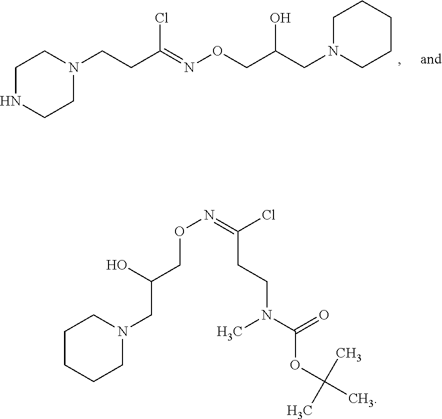 [SMILES: OC(CO/N=C(\Cl)CCN1CCNCC1)CN1CCCCC1 ; CN(CC/C(Cl)=N\OCC(O)CN1CCCCC1)C(=O)OC(C)(C)C]

19. The compound according to any one of the preceding items, wherein the compound increases glucocerebrosidase (GBA) enzyme levels and/or GBA enzyme activity.
20. The compound according to any one of the preceding items, wherein the compound is a GBA inducer.
21. The compound according to any one of the preceding items, for use in a method of increasing GBA levels and/or activity.
22. The compound for use according to any one of the preceding items, wherein said GBA activity is increased at least 1.5-fold, such as at least 2-fold, for example at least 2.5-fold, such as at least 3-fold.
23. The compound for use according to any one of the preceding items, wherein said GBA activity is increased to 50% or more of hypothetical wild-type levels, such as 50-60%, such as 60-70%, such as 70-80%, such as 80-90%, such as 90-100%, such as 100-110%, such as 110-120%, such as 120-130%, such as 130-140%, such as 140-150% of hypothetical wild-type levels.
24. The compound for use according to any one of the preceding items, wherein the EC_{1.5} of the compound is 150 μM or less, such as 140 μM or less, such as 130 μM or less, such as 120 μM or less, such as 110 μM or less, such as 100 μM or less, such as 90 μM or less, such as 80 μM or less, such as 70 μM or less, such as 60 μM or less, preferably wherein the EC_{1.5} is 50 μM or less, such as 40 μM or less, such as 30 μM or less, such as 20 μM or less, such as 10 μM or less.
25. The compound for use according to any one of the preceding items, wherein the Emax % of the compound is 80% or more, such as 100% or more, such as 120% or more, such as 140% or more, such as 160% or more, such as 180% or more, such as 200% or more, such as 220% or more, such as 240% or more, such as 260% or more, such as 280% or more, such as 300% or more.
26. A pharmaceutical composition comprising a compound as defined in any one of the preceding items, and one or more pharmaceutically acceptable adjuvants, excipients, carriers, buffers and/or diluents.
27. A method for treating a disease in a subject comprising administering a compound as defined in any one of the preceding items, wherein the disease is associated with reduced GBA levels and/or activity.
28. The method according to any one of the preceding items, wherein the disease is Parkinson's disease (PD).
29. A method of increasing the GBA activity and/or levels comprising contacting GBA with a compound as defined in any one of the preceding items.
30. Use of a compound as defined in any one of the preceding items, for the manufacture of a medicament for the treatment of Parkinson's disease (PD).

### EXAMPLES

### Example 1: Synthesis of Oximes

### Materials and Abbreviations

The abbreviations used:

| | |
|---|---|
| DCM | Dichloromethane |
| DCE | Dichloroethane |
| TFA | Trifluoroacetic acid |
| CDI | Carbonyldiimidazole |
| MeCN | Acetonitrile |
| HOAc | Acetic acid |
| MeOH | Methanol |
| H₂O | Water |
| NaBH₃CN | Sodium cyanoborohydride |
| NBS | 1-Bromo-2,5-pyrrolidinedione |
| K₂CO₃ | Potassium carbonate |
| DIPEA | N,N-Diisopropylethylamine |
| TEA | N,N-Diethylethanamine |
| HATU | 1-[Bis(dimethylamino)methylene]- |
| | 1H-1,2,3-triazolo[4,5-b]pyridinium |
| | 3-oxide hexafluorophosphate |
| DMF | Dimethylformamide |
| NaOH | Sodium hydroxide |
| THF | Tetrahydrofuran |
| LiHMDS | Lithium bis(trimethylsilyl)amide |
| DIAD | Diisopropyl azodicarboxylate |
| PPh₃ | Triphenylphosphine |
| TBAF | Tetra-n-butylammonium fluoride |
| NaBH(OAc)₃ | Sodium triacetoxyboranuide |
| MeNH₂ | Methanamine |
| Boc₂O | Di-tert-butyl dicarbonate |
| Paraform | Paraformaldehyde |
| t-BuONO | tert-Butyl nitrite |
| CuCl₂ | Copper (II) chloride |
| IPA | Propan-2-ol |
| NH₂OH*HCl | Hydroxylamine hydrochloride |
| Rac | Racemic (Some compounds may |
| | contain both known and unknown |
| | stereochemistry) |
| r.t. | Room temperature |

A straight line towards a chiral center in the schemes and structures below indicate the material is a racemate. If nothing else is noted, the structures are racemates.

The salt stoichiometry are assumptions based on normal acid base reaction considerations. The exact salt content has not been absolutely determined.

Analytical and preparative instruments used. One or more of the following instruments were used in the process of analysing composition of isolated material:

### LC/MS

### Instrument Specifications:

Agilent 1100 Series LC/MSD system with DAD\ELSD Alltech 2000ES and Agilent LC\MSD VL (G1956B), SL (G1956B) mass-spectrometer.

Agilent 1200 Series LC/MSD system with DAD\ELSD Alltech 3300 and Agilent LC\MSD G6130A, G6120B mass-spectrometer.

Agilent Technologies 1260 Infinity LC/MSD system with DAD\ELSD Alltech 3300 and Agilent LC\MSD G6120B mass-spectrometer.

Agilent Technologies 1260 Infinity II LC/MSD system with DAD\ELSD G7102A 1290 Infinity II and Agilent LC\MSD G6120B mass-spectrometer.

Agilent 1260 Series LC/MSD system with DAD\ELSD and Agilent LC\MSD (G6120B) mass-spectrometer.

UHPLC Agilent 1290 Series LC/MSD system with DAD\ELSD and Agilent LC\MSD (G6125B) mass-spectrometer.

All the LC/MS data were obtained using positive/negative mode switching.

### H-NMR

Bruker AVANCE III 400
Varian UNITY INOVA 400

For chiral analysis or separation the following instruments were used:

### Analytical Separation:

Column: Chiralpak IA (250*4.6 mm, 5 mkm)
HPLC instrument:
Agilent Technologies HPLC Systems 1200 Series with DAD Detector (G1315B).

### Preparative Separation:

Column: Chiralpak IA (250*20 mm, 5 mkm)

### HPLC Instrument:

Agilent Technologies HPLC Preparative Systems 1260 Infinity II Series with DAD Detector (G7115B).

### General Synthesis of Building Blocks

### Synthesis of 2-methyl-4-azoniaspiro[3.5]nonan-2-ol chloride

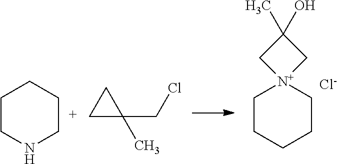

To a solution 8.5 g of piperidine in 200 ml of methanol was added 10.64 g of 2-(chloromethyl)-2-methyl-oxirane. The mixture was stirred at 25° C. 24 h and then evaporated to dryness. The residue was triturated with ether three times, and then dried in vacuo to give 16.5g crude title compound which was used as such.

### 1. General Synthesis Using Quaternary Salt

### Synthesis of N-(2-hydroxy-3-(piperidin-1-yl)propoxy)-4-methylpiperidine-4-carbimidoyl chloride dihydrochloride, ID 336

### General Scheme

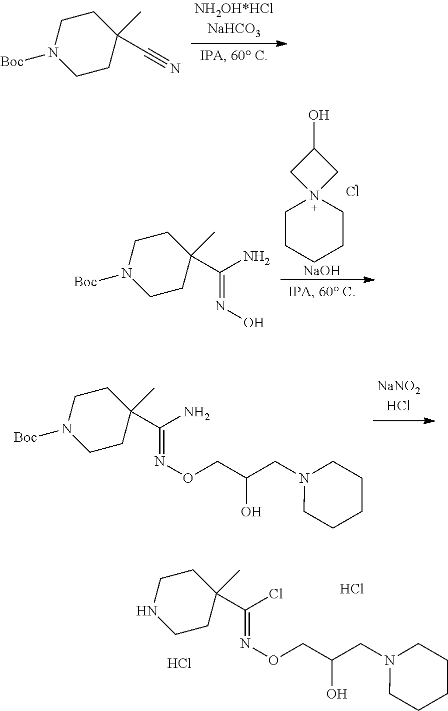

1.1 Synthesis of tert-butyl 4-(N′-hydroxycarbamimidoyl)-4-methylpiperidine-1-carboxylate

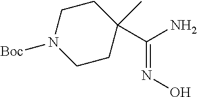 [SMILES: CC(C)(C)OC(=O)N1CCC(C)(/C(N)=N/O)CC1]

tert-Butyl 4-cyano-4-methyl-piperidine-1-carboxylate (29.0 g, 122.83 mmol) was dissolved in IPA (200 ml), after that hydroxylamine hydrochloride (12.80 g, 184.24 mmol, 1.5 eq) was added to the resulting solution, followed by the addition of sodium hydrogen carbonate (15.48 g, 184.24 mmol, 1.5 eq). The reaction mixture was then left while stirring at 60° C. overnight. After 24 hours the reaction mixture was diluted with water (500 ml). The formed solid was collected by filtration, washed with water (100 ml) and airdried to afford the title product (25 g, 75%) as white solid. LCMS [M−t-Bu H]⁺ 202.0. The product obtained was used as such without additional purification.

### 1.2 Synthesis of tert-butyl 4-(N′-(2-hydroxy-3-(piperidin-1-yl)propoxy)carbamimidoyl)-4-methylpiperidine-1-carboxylate

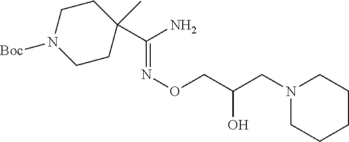 [SMILES: CC(C)(C)OC(=O)N1CCC(C)(/C(N)=N/OCC(O)CN2CCCCC2)CC1]

tert-Butyl 4-(N′-hydroxycarbamimidoyl)-4-methylpiperidine-1-carboxylate (1.5 g, 5.54 mmol, 1 eq) was dissolved in IPA (50 ml), followed by sodium hydroxide (0.222 g, 5.54 mmol, 1 eq) and 4-azoniaspiro[3.5]nonan-2-ol chloride (0.98 g, 5.54 mmol, 1 eq). The reaction mixture was then stirred for 24 hours at 50° C., after which the inorganic precipitate was removed by filtration and the filtrate collected was concentrated under reduced pressure to afford the title product (3 g, 95.15%) as a yellow oil. LCMS [M+1]⁺ 399.2.

### 1.3 Synthesis of N-(2-hydroxy-3-(piperidin-1-yl)propoxy)-4-methylpiperidine-4-carbimidoyl chloride dihydrochloride

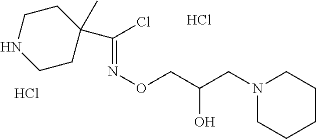 [SMILES: CC1(/C(Cl)=N/OCC(O)CN2CCCCC2)CCNCC1 ; Cl ; Cl]

tert-Butyl 4-(N′-(2-hydroxy-3-(piperidin-1-yl)propoxy)carbamimidoyl)-4-methylpiperidine-1-carboxylate, obtained in the previous experiment (3.0 g, 5.27 mmol, 1 eq) was dissolved in a mixture of acetic acid (5 ml) and aqueous hydrochloric acid (4N, 5 ml) at 0° C. Sodium nitrite (727 mg, 10.54 mmol, 2 eq) was added portion wise to the resulting solution while cooling, maintaining the temperature interval 0-5° C. After the addition was completed aqueous hydrochloric acid (4N, 5 ml) was added at 0° C. to the reaction mixture, which was left while stirring at 0° C. for 1 hour. The cooling bath was removed and the reaction mixture was allowed to warm up to room temperature and then left while stirring overnight. After 12 hours the mixture was* concentrated under reduced pressure to afford crude semi-solid product (3.1 g), which was subjected for prep HPLC purification with HCl addition to result in 386 mg (17.8%) of the title N-(2-hydroxy-3-(piperidin-1-yl)propoxy)-4-methylpiperidine-4-carbimidoyl chloride dihydrochloride as a yellow oil (METHOD A). *In an alternative work up and purification procedure, the mixture was concentrated under reduced pressure to afford crude semi-solid residue, which was diluted with 30% aqueous solution of potassium carbonate to adjust pH 10 and then extracted with DCM (3×5 ml). The organic layers were combined, dried over anhydrous sodium sulfate and filtered. The filtrate collected was concentrated under reduced pressure to afford crude oil, which was subjected for prep HPLC with trifluoroacetate addition (METHOD B LCMS [M−·Cl⁻]⁺ 282.2. ¹H NMR (Deuterium Oxide, 400 MHz): δ (ppm) 4.32-4.23 (m, 1H), 4.19-4.03 (m, 2H), 3.53-3.35 (m, 2H), 3.24-3.12 (m, 3H), 3.10-2.88 (m, 4H), 2.82 (t, J=12.1, 12.1 Hz, 1H), 2.23 (d, J=14.2 Hz, 2H), 1.88-1.74 (m, 2H), 1.75-1.57 (m, 5H), 1.44-1.30 (m, 1H), 1.17 (s, 3H).

### Example 2

### Synthesis of 4-ethyl-N-(2-hydroxy-3-(piperidin-1-yl)propoxy)piperidine-4-carbimidoyl chloride di-2,2,2-trifluoroacetate, NME200409

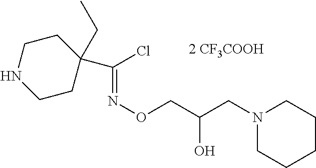 [SMILES: CCC1(/C(Cl)=N/OCC(O)CN2CCCCC2)CCNCC1 ; "2 CF3COOH"]

In a generally similar manner with non-critical variations was made 4-ethyl-N-(2-hydroxy-3-(piperidin-1-yl)propoxy)piperidine-4-carbimidoyl chloride di-2,2,2-trifluoroacetate (254 mg, 29.6%) as a yellow oil from the commercially available tert-butyl 4-cyano-4-ethylpiperidine-1-carboxylate in line with the synthesis described in 1.1 to 1.3. ¹H NMR (Deuterium Oxide, 500 MHz): δ (ppm) 4.34-4.25 (m, 1H), 4.23-4.09 (m, 2H), 3.46 (dd, J=31.0, 11.7 Hz, 2H), 3.27-3.06 (m, 4H), 3.05-2.92 (m, 3H), 2.83 (t, J=12.1, 12.1 Hz, 1H), 2.27 (d, J=14.6 Hz, 2H), 1.88-1.77 (m, 2H), 1.72-1.61 (m, 5H), 1.60-1.49 (m, 2H), 1.46-1.34 (m, 1H), 0.67 (t, J=7.4, 7.4 Hz, 3H).

### Example 3

### Synthesis of N-(2-hydroxy-3-(piperidin-1-yl)propoxy)-4-(pyridin-2-yl)piperidine-4-carbimidoyl chloride tri-2,2,2-trifluoroacetate, NME200415

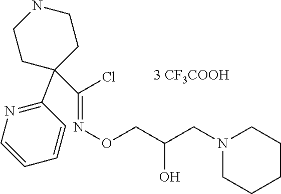 [SMILES: OC(CO/N=C(\Cl)C1(c2ccccn2)CCNCC1)CN1CCCCC1 ; "3 CF3COOH"]

In a generally similar manner with non-critical variations was made N-(2-hydroxy-3-(piperidin-1-yl)propoxy)-4-(pyridin-2-yl)piperidine-4-carbimidoyl chloride tri-2,2,2-trifluoroacetate (115 mg, 12.46%) as a yellow oil from the commercially available tert-butyl 4-cyano-4-(pyridin-2-yl)piperidine-1-carboxylate in line with the synthesis described in 1.1 to 1.3. LCMS [M−·Cl⁻]⁺ 345.0. ¹H NMR (Deuterium Oxide, 400 MHz): δ (ppm) 8.59 (d, J=4.9 Hz, 1H), 8.38 (t, J=8.4, 8.4 Hz, 1H), 7.95 (d, J=8.2 Hz, 1H), 7.81 (t, 1H), 4.35-4.24 (m, 2H), 4.16 (dd, 1H), 3.55-3.14 (m, 7H), 3.15-3.01 (m, 2H), 2.93-2.86 (m, 1H), 2.82-2.76 (m, 2H), 2.42-2.31 (m, 2H), 1.85-1.71 (m, 2H), 1.70-1.51 (m, 3H), 1.44-1.27 (m, 1H).

### Example 4

### General Scheme Preparation of Nitrile Precursors

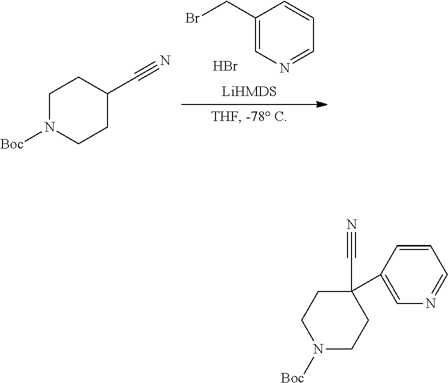 [SMILES: CC(C)(C)OC(=O)N1CCC(C#N)CC1 ; BrCc1cccnc1 ; Br ; CC(C)(C)OC(=O)N1CCC(C#N)(c2cccnc2)CC1]

### 1.4 synthesis of tert-butyl 4-cyano-4-(3-pyridylmethyl)piperidine-1-carboxylate

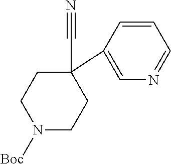 [SMILES: CC(C)(C)OC(=O)N1CCC(C#N)(c2cccnc2)CC1]

To a solution of tert-butyl 4-cyanopiperidine-1-carboxylate (1.5 g. 7.13 mmol) under nitrogen atmosphere in a round-bottomed flask was slowly added at −76° C. lithium bis(trimethylsilyl)azanide (1.0 M in THF/Ethylbenzol, 21.4 ml, 21.4 mmol). After the mixture was stirred for 2 hours at −76° C., 3-(bromomethyl)pyridine hydrobromide (1.98 g, 10.46 mmol) was added into the system. The reaction mixture was stirred for further 30 minutes and then warmed to room temperature and stirred overnight. The mixture was quenched with 50 ml saturated aqueous NH₄Cl₂ further diluted with water and extracted with EtOAc. The organic layers were washed with water and brine then dried over sodium sulfate, filtered and concentrated to afford target compound (2.6 g, 33%), which was used as such LCMS: [M+H]⁺ 302

### Synthesis of N-(2-hydroxy-3-(piperidin-1-yl)propoxy)-4-(pyridin-3-ylmethyl)piperidine-4-carbimidoyl chloride tri-2,2,2-trifluoroacetate, NME200419

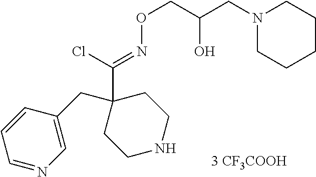 [SMILES: OC(CO/N=C(\Cl)C1(Cc2cccnc2)CCNCC1)CN1CCCCC1]

In a generally similar manner with non-critical variations was made N-(2-hydroxy-3-(piperidin-1-yl)propoxy)-4-(pyridin-3-ylmethyl)piperidine-4-carbimidoyl chloride tri-2,2,2-trifluoroacetate (720 mg, 33.19%) as a yellow oil using tert-butyl 4-cyano-4-(3-pyridylmethyl)piperidine-1-carboxylate instead of tert-butyl 4-cyanopiperidine-1-carboxylate in line with the synthesis described in 1.1 to 1.3, and 1.4 respectively LCMS [M+1]⁺ 396.2. ¹H NMR (Chloroform-d, 400 MHz): δ (ppm) 8.48 (d, J=4.5 Hz, 1H), 8.37 (s, 1H), 7.44 (d, 1H), 7.23-7.17 (m, 1H), 4.46-4.31 (m, 1H), 4.05 (d, J=13.2 Hz, 1H), 3.68 (dd, J=11.6, 5.7 Hz, 1H), 3.38-3.22 (m, 2H), 3.16-2.98 (m, 2H), 2.88-2.74 (m, 2H), 2.66-2.24 (m, 6H), 2.27-2.15 (m, 2H), 1.91 (t, J=14.4, 14.4 Hz, 2H), 1.64-1.51 (m, 4H), 1.46-1.34 (m, 2H).

### Example 5

### Synthesis of 3-(dimethylamino)-N-(2-hydroxy-3-(piperidin-1-yl)propoxy)propanimidoyl chloride, NME200446

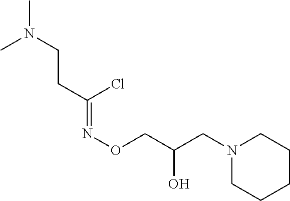 [SMILES: CN(C)CC/C(Cl)=N/OCC(O)CN1CCCCC1]

In a generally similar manner with non-critical variations was made 3-(dimethylamino)-N-(2-hydroxy-3-(piperidin-1-yl)propoxy)propanimidoyl chloride (445.2 mg, 41.56%) as a yellow oil from the commercially available 3-(dimethylamino)propanenitrile in line with the synthesis described in 1.1 to 1.3. LCMS [M+1]⁺ 292.2. ¹H NMR (DMSO-d₆, 400 MHz): δ (ppm) 4.67-4.58 (m, 1H), 4.08-4.00 (m, 1H), 3.99-3.92 (m, 1H), 3.88-3.75 (m, 1H), 2.64-2.56 (m, 2H), 2.49-2.45 (m, 2H), 2.42-2.31 (m, 4H), 2.29-2.21 (m, 2H), 2.13 (s, 6H), 1.52-1.42 (m, 4H), 1.39-1.28 (m, 2H).

### Example 6

### Synthesis of N-(2-hydroxy-3-(piperidin-1-yl)propoxy)-4-(2-methoxyethyl)piperidine-4-carbimidoyl chloride di-2,2,2-trifluoroacetate, NME200448

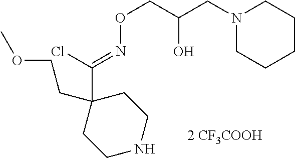 [SMILES: COCCC1(/C(Cl)=N/OCC(O)CN2CCCCC2)CCNCC1]

In a generally similar manner with non-critical variations was made N-(2-hydroxy-3-(piperidin-1-yl)propoxy)-4-(2-methoxyethyl)piperidine-4-carbimidoyl chloride di-2,2,2-trifluoroacetate (26.5 mg, 6.63%) as a yellow oil from the commercially available tert-butyl 4-cyano-4-(2-methoxyethyl)piperidine-1-carboxylate in line with the synthesis described in 1.1 to 1.3. LCMS [M−·Cl⁻]⁺ 326.2. ¹H NMR (Methanol-d₄, 400 MHz): δ (ppm) 4.38-4.28 (m, 1H), 4.22 (d, J=4.9 Hz, 2H), 3.64-3.51 (m, 2H), 3.40 (t, J=6.1, 6.1 Hz, 2H), 3.34-3.21 (m, 8H), 3.19-2.96 (m, 4H), 2.39 (d, J=14.5 Hz, 2H), 2.00-1.67 (m, 8H), 1.62-1.47 (m, 1H).

### Example 7

### Synthesis of N-(2-hydroxy-3-(piperidin-1-yl)propoxy)pyridazine-4-carbimidoyl bromide, NME200452

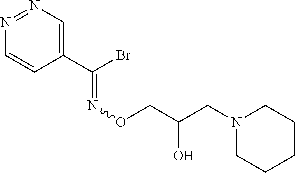 [SMILES: OC(CON=C(Br)c1ccnnc1)CN1CCCCC1]

In a generally similar manner with non-critical variations was made N-(2-hydroxy-3-(piperidin-1-yl)propoxy)pyridazine-4-carbimidoyl bromide (159.9 mg, 12.36%) as a brown oil from the commercially available pyridazine-4-carbonitrile in line with the synthesis described in 1.1 to 1.3 but using hydrobromic acid instead of hydrochloric acid in experimental procedure 1.3. LCMS [M+1]⁺ 345.2. ¹H NMR (Chloroform-d, 400 MHz): δ (ppm) 9.57 (s, 1H), 9.24 (d, J=5.3 Hz, 1H), 7.78 (d, J=7.7 Hz, 1H), 4.39 (d, J=5.0 Hz, 2H), 4.12-4.02 (m, 1H), 3.53 (s, 1H), 2.65-2.55 (m, 2H), 2.46-2.40 (m, 2H), 2.38-2.28 (m, 2H), 1.63-1.50 (m, 4H), 1.47-1.38 (m, 2H).

### Example 8

### Synthesis of N-(2-hydroxy-3-(piperidin-1-yl)propoxy)-2-methylthiazole-5-carbimidoyl chloride, NME200453

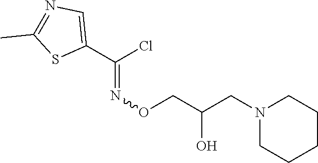 [SMILES: Cc1ncc(C(Cl)=NOCC(O)CN2CCCCC2)s1]

In a generally similar manner with non-critical variations was made N-(2-hydroxy-3-(piperidin-1-yl)propoxy)-2-methylthiazole-5-carbimidoyl chloride (136 mg, 21.28%) as a yellow oil from the commercially available 2-methyl-1,3-thiazole-5-carbonitrile in line with the synthesis described in 1.1 to 1.3. LCMS [M+1]⁺ 318.2. ¹H NMR (Chloroform-d, 400 MHz): δ (ppm) 7.93 (s, 1H), 7.24 (s, 1H), 4.29-4.16 (m, 2H), 4.07-3.99 (m, 1H), 2.68 (s, 3H), 2.62-2.51 (m, 2H), 2.42-2.27 (m, 4H), 1.63-1.51 (m, 4H), 1.47-1.38 (m, 2H).

### Example 9

### Synthesis of 6-chloro-N-(2-hydroxy-3-(piperidin-1-yl)propoxy)nicotinimidoyl chloride, NME200466

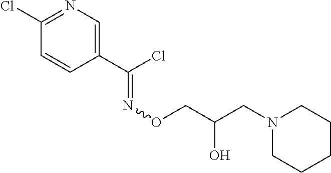 [SMILES: OC(CON=C(Cl)c1ccc(Cl)nc1)CN1CCCCC1]

In a generally similar manner with non-critical variations was made 6-chloro-N-(2-hydroxy-3-(piperidin-1-yl)propoxy)nicotinimidoyl chloride (239 mg, 23.82%) as a yellow oil from the commercially available 6-chloronicotinonitrile in line with the synthesis described in 1.1 to 1.3. LCMS [M+1]⁺ 332.2. ¹H NMR (Chloroform-d, 400 MHz): δ (ppm) 8.80 (d, J=2.3 Hz, 1H), 8.05 (dd, J=8.4, 2.4 Hz, 1H), 7.34 (d, J=8.4 Hz, 1H), 4.31-4.25 (m, 2H), 4.08-4.01 (m, 1H), 3.47 (s, 1H), 2.66-2.57 (m, 2H), 2.40-2.29 (m, 4H), 1.60-1.53 (m, 4H), 1.44-1.39 (m, 2H).

### Example 10

### Synthesis of N-(2-hydroxy-3-(piperidin-1-yl)propoxy)-3-methylisoxazole-5-carbimidoyl chloride, NME200475

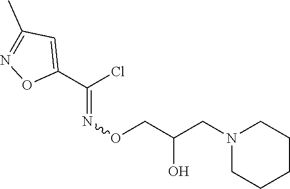 [SMILES: Cc1cc(C(Cl)=NOCC(O)CN2CCCCC2)on1]

In a generally similar manner with non-critical variations was made N-(2-hydroxy-3-(piperidin-1-yl)propoxy)-3-methylisoxazole-5-carbimidoyl chloride (360 mg, 24.46%) as a yellow oil from the commercially available 3-methylisoxazole-5-carbonitrile in line with the synthesis described in 1.1 to 1.3. LCMS [M+1]⁺ 302.2. ¹H NMR (Chloroform-d, 400 MHz): δ (ppm) 6.50 (s, 1H), 4.31 (d, J=5.0 Hz, 2H), 4.09-4.01 (m, 1H), 3.76 (s, 1H), 2.65-2.54 (m, 2H), 2.42-2.25 (m, 7H), 1.64-1.48 (m, 4H), 1.47-1.36 (m, 2H).

### Example 11

### Synthesis of 6-chloro-N-(2-hydroxy-3-(piperidin-1-yl)propoxy)-4-methylnicotinimidoyl chloride, NME200501

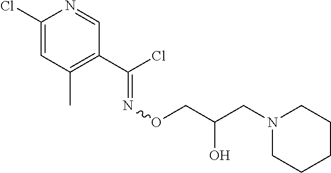 [SMILES: Cc1cc(Cl)ncc1C(Cl)=NOCC(O)CN1CCCCC1]

In a generally similar manner with non-critical variations was made 6-chloro-N-(2-hydroxy-3-(piperidin-1-yl)propoxy)-4-methylnicotinimidoyl chloride (66.3 mg, 2.48%) as a yellow oil from the commercially available 6-chloro-4-methylnicotinonitrile in line with the synthesis described in 1.1 to 1.3. LCMS [M+1]⁺ 346.2. ¹H NMR (Chloroform-d, 400 MHz): δ (ppm) 8.44 (s, 1H), 7.20 (s, 1H), 4.26 (d, J=5.6 Hz, 2H), 4.08-4.00 (m, 1H), 3.76 (s, 1H), 2.62-2.55 (m, 2H), 2.44 (s, 3H), 2.41-2.29 (m, 4H), 1.62-1.52 (m, 4H), 1.46-1.38 (m, 2H).

### Example 12

### Synthesis of 6-chloro-N-(2-hydroxy-3-(piperidin-1-yl)propoxy)nicotinimidoyl bromide, NME200504

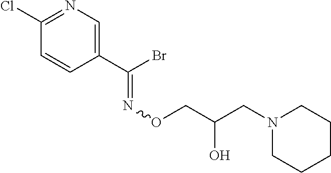 [SMILES: OC(CON=C(Br)c1ccc(Cl)nc1)CN1CCCCC1]

In a generally similar manner with non-critical variations was made 6-chloro-N-(2-hydroxy-3-(piperidin-1-yl)propoxy)nicotinimidoyl bromide (421.8 mg, 18.49%) as a yellow oil from the commercially available 6-chloronicotinonitrile in line with the synthesis described in 1.1 to 1.3 but using hydrobromic acid instead of hydrochloric acid in experimental procedure 1.3. LCMS [M+1]⁺ 376.2. ¹H NMR (Chloroform-d, 500 MHz): δ (ppm) 8.82 (s, 1H), 8.07 (d, J=10.6 Hz, 1H), 7.36 (d, J=8.4 Hz, 1H), 4.48-4.27 (m, 2H), 4.13-4.04 (m, 1H), 2.74-2.54 (m, 2H), 2.51-2.24 (m, 4H), 1.64-1.52 (m, 4H), 1.49-1.36 (m, 2H).

### Example 13

### Synthesis of 3-(dimethylamino)-N-(2-hydroxy-3-(piperidin-1-yl)propoxy)propanimidoyl bromide, NME200532

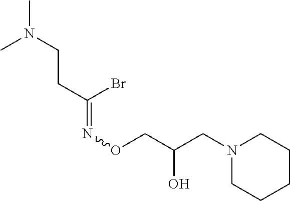 [SMILES: CN(C)CCC(Br)=NOCC(O)CN1CCCCC1]

In a generally similar manner with non-critical variations was made 3-(dimethylamino)-N-(2-hydroxy-3-(piperidin-1-yl)propoxy)propanimidoyl bromide (123.6 mg, 16.69%) as a yellow oil from the commercially available 3-(dimethylamino)propanenitrile in line with the synthesis described in 1.1 to 1.3 but using hydrobromic acid instead of hydrochloric acid in experimental procedure 1.3. LCMS [M+1]⁺ 337.8. ¹H NMR (DMSO-d₆, 400 MHz): δ (ppm) 4.64 (s, 1H), 4.15-4.04 (m, 1H), 4.03-3.93 (m, 1H), 3.89-3.79 (m, 1H), 2.69 (t, J=6.7, 6.7 Hz, 2H), 2.49-2.43 (m, 2H), 2.40-2.20 (m, 6H), 2.14 (s, 6H), 1.54-1.42 (m, 4H), 1.40-1.30 (m, 2H).

### Example 14

### Synthesis of N-(2-hydroxy-3-(piperidin-1-yl)propoxy)-4-(pyridin-3-yl)piperidine-4-carbimidoyl chloride tri-2,2,2-trifluoroacetate, NME200492

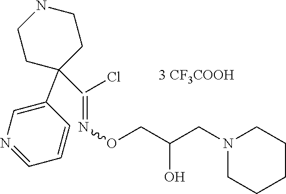

In a generally similar manner with non-critical variations was made N-(2-hydroxy-3-(piperidin-1-yl)propoxy)-4-(pyridin-3-yl)piperidine-4-carbimidoyl chloride tri-2,2,2-trifluoroacetate (116.4 mg, 17.22%) as a yellow oil from the commercially available tert-butyl 4-cyano-4-(pyridin-3-yl)piperidine-1-carboxylate in line with the synthesis described in 1.1 to 1.3. LCMS [M−·Cl⁻]⁺ 345.4. ¹H NMR (Methanol-d₄, 400 MHz): δ (ppm) 8.82 (s, 1H), 8.72 (d, J=4.2 Hz, 1H), 8.32 (d, J=8.1 Hz, 1H), 7.82 (dd, J=8.0, 5.3 Hz, 1H), 4.40-4.28 (m, 2H), 3.64-3.52 (m, 2H), 3.43-3.26 (m, 8H), 3.25-3.13 (m, 2H), 3.07-2.93 (m, 2H), 2.85 (d, J=14.9 Hz, 2H), 2.42 (t, J=12.8, 12.8 Hz, 2H), 1.96-1.87 (m, 2H), 1.85-1.75 (m, 2H), 1.59-1.47 (m, 1H).

### Example 15

### Synthesis of N-(2-hydroxy-3-(piperidin-1-yl)propoxy)-4-((6-methylpyridin-3-yl)methyl)piperidine-4-carbimidoyl chloride tri-2,2,2-trifluoroacetate, NME200493

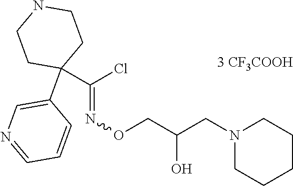

In a generally similar manner with non-critical variations was made N-(2-hydroxy-3-(piperidin-1-yl)propoxy)-4-((6-methylpyridin-3-yl)methyl)piperidine-4-carbimidoyl chloride tri-2,2,2-trifluoroacetate (108.2 mg, 27.64%) as a pale brown oil in line with the synthesis described in 3.1-3.3, but using 5-(bromomethyl)-2-methylpyridine hydrobromide instead of 3-(bromomethyl)-pyridine hydrobromide in experimental procedure 1.4. LCMS [M+1]⁺ 410.2. ¹H NMR (Deuterium Oxide, 400 MHz): δ (ppm) 8.26 (s, 1H), 8.06 (d, J=9.6 Hz, 1H), 7.68 (d, J=8.3 Hz, 1H), 4.18-4.06 (m, 1H), 4.02-3.86 (m, 2H), 3.40 (t, J=11.0, 11.0 Hz, 2H), 3.24 (d, J=13.2 Hz, 2H), 3.10-2.83 (m, 7H), 2.79 (t, J=12.0, 12.0 Hz, 1H), 2.60 (s, 3H), 2.22 (d, J=14.5 Hz, 2H), 1.91-1.70 (m, 4H), 1.73-1.58 (m, 3H), 1.41-1.29 (m, 1H).

### Example 16

### Synthesis of N-(2-hydroxy-3-(piperidin-1-yl)propoxy)-4-((6-methoxy-2-methylpyridin-3-yl)methyl)piperidine-4-carbimidoyl chloride tri-2,2,2-trifluoroacetate, NME200524

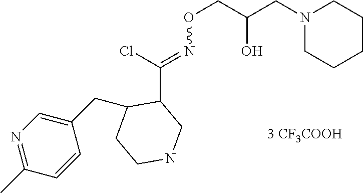 [SMILES: Cc1ccc(CC2CCNCC2C(Cl)=NOCC(O)CN2CCCCC2)cn1]

In a generally similar manner with non-critical variations was made N-(2-hydroxy-3-(piperidin-1-yl)propoxy)-4-((6-methoxy-2-methylpyridin-3-yl)methyl)piperidine-4-carbimidoyl chloride tri-2,2,2-trifluoroacetate (102.5 mg, 18.89%) as a pale brown oil in line with the synthesis described in 1.1-1.3, but using 3-(chloromethyl)-6-methoxy-2-methylpyridine instead of 3-(bromomethyl)-pyridine hydrobromide in experimental procedure 1.4. LCMS [M−·Cl⁻]⁺ 403.2. ¹H NMR (Deuterium Oxide, 400 MHz): δ (ppm) 7.95 (d, J=9.0 Hz, 1H), 7.13 (d, J=9.0 Hz, 1H), 4.20-4.12 (m, 1H), 4.07-3.90 (m, 5H), 3.40 (t, J=11.3, 11.3 Hz, 2H), 3.23 (d, J=13.0 Hz, 2H), 3.10-2.98 (m, 2H), 2.96-2.74 (m, 6H), 2.42 (s, 3H), 2.27 (d, J=14.7 Hz, 2H), 1.85-1.73 (m, 4H), 1.70-1.58 (m, 3H), 1.39-1.26 (m, 1H).

### Example 17

### Synthesis of N-(2-hydroxy-3-(piperidin-1-yl)propoxy)-4-((6-methoxy-4-methylpyridin-3-yl)methyl)piperidine-4-carbimidoyl chloride, NME200525

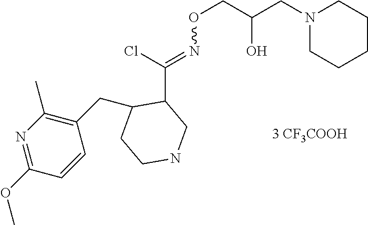 [SMILES: COc1ccc(CC2CCNCC2C(Cl)=NOCC(O)CN2CCCCC2)c(C)n1]

In a generally similar manner with non-critical variations was made N-(2-hydroxy-3-(piperidin-1-yl)propoxy)-4-((6-methoxy-4-methylpyridin-3-yl)methyl)piperidine-4-carbimidoyl chloride (62.6 mg, 11.42%) as a yellow oil in line with the synthesis described in 1.1-1.3, but using 5-(chloromethyl)-2-methoxy-4-methylpyridine instead of 3-(bromomethyl)-pyridine hydrobromide in experimental procedure 1.4. LCMS [M+1]⁺ 439.2. ¹H NMR (Deuterium Oxide, 400 MHz): δ (ppm) 7.77 (s, 1H), 7.22 (s, 1H), 4.23-3.92 (m, 7H), 3.41 (t, J=12.8, 12.8 Hz, 2H), 3.27-3.20 (m, 2H), 3.10-3.01 (m, 2H), 2.94-2.86 (m, 4H), 2.83-2.76 (m, 1H), 2.38 (s, 3H), 2.29 (d, J=14.4 Hz, 2H), 1.89-1.75 (m, 4H), 1.71-1.56 (m, 3H), 1.42-1.28 (m, 1H).

### Example 18

### Synthesis of N-(2-hydroxy-3-(piperidin-1-yl)propoxy)-4-(pyridin-2-yl)piperidine-4-carbimidoyl chloride tri-2,2,2-trifluoroacetate, NME200534

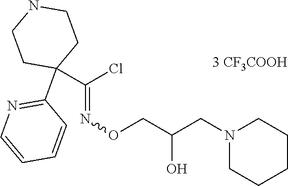

In a generally similar manner with non-critical variations was made N-(2-hydroxy-3-(piperidin-1-yl)propoxy)-4-(pyridin-2-yl)piperidine-4-carbimidoyl chloride tri-2,2,2-trifluoroacetate (115 mg, 12.46%) as a yellow oil from the commercially available tert-butyl 4-cyano-4-(pyridin-2-yl)piperidine-1-carboxylate in line with the synthesis described in 1.1 to 1.3. LCMS [M−·Cl⁻]⁺ 345.2. ¹H NMR (Deuterium Oxide, 400 MHz): δ (ppm) 8.59 (d, J=4.9 Hz, 1H), 8.38 (t, J=8.4, 8.4 Hz, 1H), 7.95 (d, J=8.2 Hz, 1H), 7.86-7.78 (m, 1H), 4.34-4.22 (m, 2H), 4.19-4.11 (m, 1H), 3.47-3.21 (m, 6H), 3.15-2.98 (m, 2H), 2.96-2.86 (m, 1H), 2.85-2.75 (m, 3H), 2.36 (t, J=14.8, 14.8 Hz, 2H), 1.86-1.71 (m, 2H), 1.69-1.54 (m, 3H), 1.42-1.29 (m, 1H).

### Example 19

### Synthesis of 4-(3-chloropyridin-4-yl)-N-(2-hydroxy-3-(piperidin-1-yl)propoxy)piperidine-4-carbimidoyl chloride tri-2,2,2-trifluoroacetate, NME200535

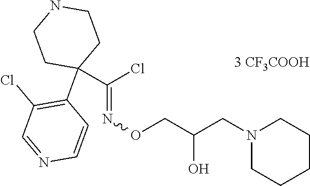

In a generally similar manner with non-critical variations was made 4-(3-chloropyridin-4-yl)-N-(2-hydroxy-3-(piperidin-1-yl)propoxy)piperidine-4-carbimidoyl chloride tri-2,2,2-trifluoroacetate (65 mg, 13.72%) as a yellow oil from the starting material tert-butyl 4-(3-chloropyridin-4-yl)-4-cyanopiperidine-1-carboxylate in line with the synthesis described in 1.1 to 1.3. The synthesis of the starting material described above. LCMS [M+1]⁺ 416.0. ¹H NMR (Deuterium Oxide, 400 MHz): δ (ppm) 8.59 (s, 1H), 8.52 (d, J=5.3 Hz, 1H), 7.72 (d, 1H), 4.33-4.19 (m, 3H), 3.51-3.26 (m, 6H), 3.20-3.02 (m, 2H), 2.95-2.76 (m, 4H), 2.41-2.25 (m, 2H), 1.87-1.75 (m, 2H), 1.72-1.56 (m, 3H), 1.46-1.30 (m, 1H).

### Example 20

### Synthesis of N-(2-hydroxy-3-(piperidin-1-yl)propoxy)-3-(pyrrolidin-1-yl)propanimidoyl chloride, NME200562

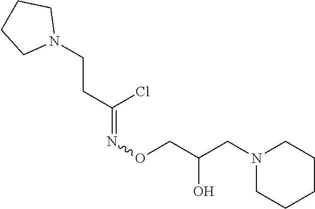 [SMILES: OC(CON=C(Cl)CCN1CCCC1)CN1CCCCC1]

In a generally similar manner with non-critical variations was made N-(2-hydroxy-3-(piperidin-1-yl)propoxy)-3-(pyrrolidin-1-yl)propanimidoyl chloride (168.1 mg, 31.57%) as a yellow oil from the commercially available 3-(pyrrolidin-1-yl)propanenitrile in line with the synthesis described in 1.1 to 1.3. LCMS [M+1]⁺ 318.2. ¹H NMR (Methanol-d₄, 400 MHz): δ (ppm) 4.07-4.01 (m, 2H), 3.32-3.25 (m, 3H), 2.84-2.65 (m, 4H), 2.61-2.56 (m, 3H), 2.53-2.41 (m, 5H), 1.92-1.71 (m, 4H), 1.64-1.55 (m, 4H), 1.50-1.41 (m, 2H).

### Example 21

### Synthesis of N-(2-hydroxy-3-(piperidin-1-yl)propoxy)-3-(piperidin-1-yl)propanimidoyl chloride, NME200563

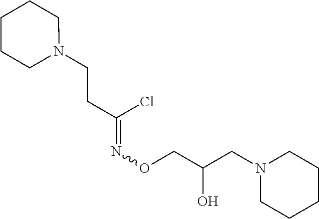 [SMILES: OC(CON=C(Cl)CCN1CCCCC1)CN1CCCCC1]

In a generally similar manner with non-critical variations was made N-(2-hydroxy-3-(piperidin-1-yl)propoxy)-3-(piperidin-1-yl)propanimidoyl chloride (119.3 mg, 23.71%) as a yellow oil from the commercially available 3-(piperidin-1-yl)propanenitrile in line with the synthesis described in 1.1 to 1.3. LCMS [M+1]⁺ 332.2. ¹H NMR (Methanol-da, 400 MHz): δ (ppm) 4.14-3.95 (m, 3H), 3.30 (s, 2H), 2.75-2.60 (m, 4H), 2.51-2.37 (m, 8H), 1.68-1.52 (m, 8H), 1.50-1.36 (m, 4H).

### Example 22

### Synthesis of 6-chloro-N-(2-hydroxy-2-methyl-3-(piperidin-1-yl)propoxy)nicotinimidoyl chloride, NME200502

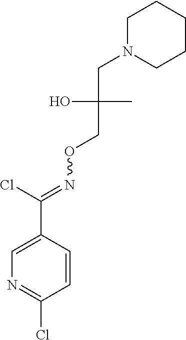 [SMILES: CC(O)(CON=C(Cl)c1ccc(Cl)nc1)CN1CCCCC1]

In a generally similar manner with non-critical variations was made 6-chloro-N-(2-hydroxy-2-methyl-3-(piperidin-1-yl)propoxy)nicotinimidoyl chloride (433.3 mg, 38.85%) as a yellow oil from the commercially available 6-chloronicotinonitrile in line with the synthesis described in 1.1 to 1.3 but using 2-hydroxy-2-methyl-4-azaspiro[3.5]nonan-4-ium chloride described above instead of 2-hydroxy-4-azaspiro[3.5]nonan-4-ium chloride in experimental procedure 1.2. LCMS [M+1]⁺ 346.2. ¹H NMR (Chloroform-d, 400 MHz): δ (ppm) 8.80 (s, 1H), 8.05 (d, 1H), 7.33 (d, J=8.4 Hz, 1H), 4.20-4.11 (m, 2H), 3.75 (s, 1H), 2.61-2.47 (m, 5H), 2.24 (d, J=13.9 Hz, 1H), 1.58-1.50 (m, 4H), 1.45-1.35 (m, 2H), 1.20 (s, 3H).

### Example 23

### Synthesis of tert-butyl (3-chloro-3-((2-hydroxy-3-(piperidin-1-yl)propoxy)imino)propyl)(methyl)carbamate, NME200599 General scheme

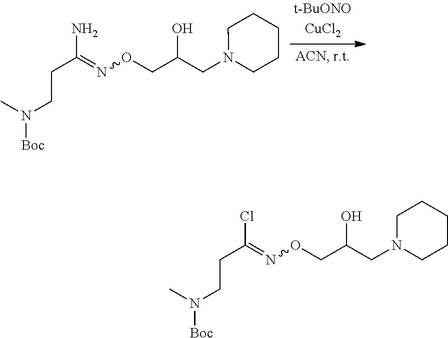 [SMILES: CN(CCC(N)=NOCC(O)CN1CCCCC1)C(=O)OC(C)(C)C ; CN(CCC(Cl)=NOCC(O)CN1CCCCC1)C(=O)OC(C)(C)C]

3.4 Synthesis of tert-butyl (3-chloro-3-((2-hydroxy-3-(piperidin-1-yl)propoxy)imino)propyl)(methyl)carbamate

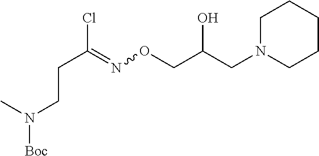 [SMILES: CN(CCC(Cl)=NOCC(O)CN1CCCCC1)C(=O)OC(C)(C)C]

tert-Butyl N-[3-amino-3-[2-hydroxy-3-(1-piperidyl)propoxy]imino-propyl]-N-methyl-carbamate, obtained in a similar manner with non-critical variations from commercially available tert-butyl (2-cyanoethyl)(methyl)carbamate in line with the synthesis described in 1.1 to 1.2 (2 g, 3.12 mmol, 1 eq) was dissolved in acetonitrile (50 ml), after that tert-butyl nitrite (0.97 g, 9.37 mmol, 3 eq) was added to the resulting solution, followed by the addition of CuCl₂ (1.26 g, 9.37 mmol, 3 eq). The reaction mixture was left while stirring at room temperature for 2 days in the dark. After 48 hours the reaction mixture was concentrated under reduced pressure, diluted with 4.0 M aqueous solution of sodium carbonate (50 ml), and extracted with ethyl acetate (2×30 ml). The organic layers were combined, dried over anhydrous sodium sulfate, and filtered. The filtrate collected was concentrated under reduced pressure to afford crude yellow oil (1 g), which was subjected to prep HPLC purification to give the title product (95 mg, 7.6%) as a pink oil. LCMS [M+1]⁺ 378.4. ¹H NMR (Chloroform-d, 400 MHz): δ (ppm) 4.20-4.02 (m, 2H), 4.01-3.88 (m, 1H), 3.58-3.31 (m, 2H), 2.84 (s, 3H), 2.74-2.47 (m, 4H), 2.43-2.27 (m, 4H), 1.71-1.45 (m, 6H), 1.43 (s, 9H).

### 1. Approach Through Usage of Nosyl Epoxide

### Example 1

### Synthesis of N-(3-(4-fluoropiperidin-1-yl)-2-hydroxypropoxy)-4-methylpiperidine-4-carbimidoyl chloride di-2,2,2-trifluoroacetate, NME200399 General Scheme

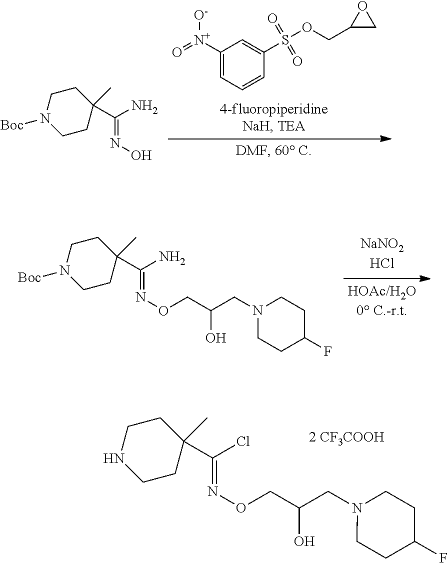 [SMILES: CC(C)(C)OC(=O)N1CCC(C)(/C(N)=N/O)CC1 ; O=[N+]([O-])c1cccc(S(=O)(=O)OCC2CO2)c1 ; CC(C)(C)OC(=O)N1CCC(C)(/C(N)=N/OCC(O)CN2CCC(F)CC2)CC1 ; CC1(/C(Cl)=N/OCC(O)CN2CCC(F)CC2)CCNCC1 ; "2 CF3COOH"]

### 1.1 Synthesis of tert-butyl 4-(N′-(3-(4-fluoropiperidin-1-yl)-2-hydroxypropoxy)carbamimidoyl)-4-methylpiperidine-1-carboxylate

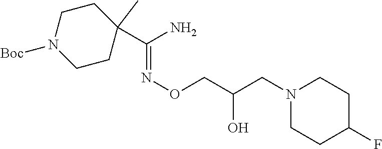 [SMILES: CC(C)(C)OC(=O)N1CCC(C)(/C(N)=N/OCC(O)CN2CCC(F)CC2)CC1]

A suspension of tert-Butyl 4-(N′-hydroxycarbamimidoyl)-4-methylpiperidine-1-carboxylate (2 g, 7.77 mmol, 1 eq) in dry DMF (5 ml) was cooled down to 0° C. Sodium hydride in mineral oil, 60% (0.311 g, 7.77 mmol, 1 eq) was added, and the the reaction mixture was stirred at 0° C. for 30 minutes. Then oxiran-2-ylmethyl 3-nitrobenzenesulfonate (2.015 g, 7.77 mmol, 1 eq) dissolved in dry DMF (5 ml) was added the mixture was allowed to warm up to room temperature and stirred for additional 2 hours. 4-Fluoropiperidine hydrochloride (1.085 g, 7.77 mmol, 1 eq) and N,N-diethylethanamine (0.787 g, 7.77 mmol, 1 eq) in dry DMF (5 ml) was added to the reaction mixture in a drop-wise manner. The reaction mass was heated up to 60° C. and left while stirring for 48 hours after which the it was concentrated under reduced pressure, diluted with distilled water (20 ml) and extracted with DCM (3×15 ml). The organic layers were combined and washed saturated solution of sodium hydrocarbonate (aq, 2×15 ml), dried over anhydrous sodium sulfate, filtered. The collected was concentrated under reduced pressure to afford brown oil (3.5 g). The material obtained was subjected for flash chromatography purification (Companion combiflash, 80 g SiO₂, acetonitrile/methanol with methanol from 0-25%, flow rate=60 mL/min) to afford the title product (1.515 g, 44.5%) as pale brown oil of satisfactorily. LCMS [M+1]⁺ 417.2.

### 1.2 Synthesis of N-(3-(4-fluoropiperidin-1-yl)-2-hydroxypropoxy)-4-methylpiperidine-4-carbimidoyl chloride di-2,2,2-trifluoroacetate

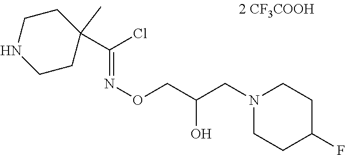 [SMILES: CC1(/C(Cl)=N/OCC(O)CN2CCC(F)CC2)CCNCC1]

tert-Butyl 4-[N′-[3-(4-fluoro-1-piperidyl)-2-hydroxy-propoxy]carbamimidoyl]-4-methyl-piperidine-1-carboxylate (500 mg, 1.14 mmol, 1 eq) was dissolved in distilled water (2 ml) and acetic acid (1 ml). The resulting solution was cooled down to 0° C. and aqueous hydrochloric acid, 30% (0.603 ml, 692.97 mg, 5.7 mmol, 5 eq) was added drop-wise to the reaction mixture, followed by a slow addition a solution of sodium nitrite (157.37 mg, 2.28 mmol, 2 eq) in distilled water (1 ml). The reaction mixture was left while stirring at 0° C. for 2 hours, and then allowed to warm up to room temperature. Aqueous hydrochloric acid, 30% (0.603 ml, 692.97 mg, 5.7 mmol, 5 eq) was added to the reaction mixture after 6 hours and then left while stirring over night at room temperature and reduced in volume and subjected for prep HPLC to afford the title product (290.7 mg, 42.95%) as a yellow oil. LCMS [M−·Cl⁻]⁺ 301.0. ¹H NMR (DMSO-d₆, 400 MHz): δ (ppm) 9.54 (s, 1H), 8.75 (s, 1H), 8.61 (s, 1H), 5.13-4.68 (m, 1H), 4.31-4.17 (m, 1H), 4.16-4.04 (m, 2H), 3.61-3.34 (m, 2H), 3.34-3.01 (m, 7H), 3.01-2.87 (m, 2H), 2.22-1.90 (m, 6H), 1.75 (t, J=13.3, 13.3 Hz, 2H), 1.24 (s, 3H).

### Example 2

### Synthesis of N-(2-hydroxy-3-(4-methylpiperazin-1-yl)propoxy)-4-methylpiperidine-4-carbimidoyl chloride tri-2,2,2-trifluoroacetate, NME200402

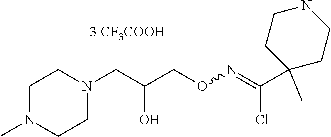 [SMILES: CN1CCN(CC(O)CON=C(Cl)C2(C)CCNCC2)CC1]

In a generally similar manner with non-critical variations was made N-(2-hydroxy-3-(4-methylpiperazin-1-yl)propoxy)-4-methylpiperidine-4-carbimidoyl chloride tri-2,2,2-trifluoroacetate (420 mg, 51.47%) as a yellow oil from the commercially available tert-butyl 4-(N′-hydroxycarbamimidoyl)-4-methylpiperidine-1-carboxylate in line with the synthesis described in 4.1 to 4.2 but using 1-methylpiperazine instead of 4-fluoropiperidine hydrochloride in experimental procedure 4.1. LCMS [M−·Cl⁻]⁺ 298.0. ¹H NMR (DMSO-d₆, 400 MHz): δ (ppm) 8.80 (s, 1H), 8.67 (s, 1H), 4.25-4.01 (m, 3H), 3.69-3.34 (m, 5H), 3.29-3.12 (m, 4H), 3.13-3.06 (m, 1H), 3.06-2.84 (m, 4H), 2.55-2.49 (m, 6H), 2.22-2.07 (m, 2H), 1.83-1.62 (m, 2H), 1.24 (s, 3H).

### Example 3

### Synthesis of N-(3-(3-azabicyclo[3.1.0]hexan-3-yl)-2-hydroxypropoxy)-4-methylpiperidine-4-carbimidoyl chloride di-2,2,2-trifluoroacetate, NME200403

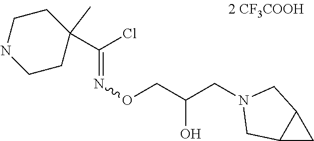 [SMILES: CC1(C(Cl)=NOCC(O)CN2CC3CC3C2)CCNCC1]

In a generally similar manner with non-critical variations was made N-(3-(3-azabicyclo[3.1.0]hexan-3-yl)-2-hydroxypropoxy)-4-methylpiperidine-4-carbimidoyl chloride di-2,2,2-trifluoroacetate (89.5 mg, 15.50%) as a yellow oil from the commercially available tert-butyl 4-(N′-hydroxycarbamimidoyl)-4-methylpiperidine-1-carboxylate in line with the synthesis described in 2.1 to 2.2 but using 3-azabicyclo[3.1.0]hexane hydrochloride instead of 4-fluoropiperidine hydrochloride in experimental procedure 2.1. LCMS [M−·Cl⁻]⁺ 280.0. ¹H NMR (DMSO-d₆, 400 MHz): δ (ppm) 9.59 (s, 1H), 8.76 (s, 1H), 8.64 (s, 1H), 4.19-3.84 (m, 4H), 3.65-3.55 (m, 2H), 3.44-3.32 (m, 2H), 3.27-3.21 (m, 1H), 3.05-2.84 (m, 3H), 2.15-2.02 (m, 2H), 1.90-1.67 (m, 5H), 1.24 (s, 4H), 0.88-0.77 (m, 1H), 0.72-0.59 (m, 1H).

### Example 4

### Synthesis of N-(3-(2-azabicyclo[2.2.1]heptan-2-yl)-2-hydroxypropoxy)-4-methylpiperidine-4-carbimidoyl chloride di-2,2,2-trifluoroacetate, NME200410

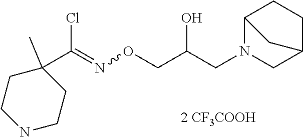 [SMILES: CC1(C(Cl)=NOCC(O)CN2CC3CCC2C3)CCNCC1]

In a generally similar manner with non-critical variations was made N-(3-(2-azabicyclo[2.2.1]heptan-2-yl)-2-hydroxypropoxy)-4-methylpiperidine-4-carbimidoyl chloride di-2,2,2-trifluoroacetate (104 mg, 19.97%) as a pale brown oil from the commercially available tert-butyl 4-(N′-hydroxycarbamimidoyl)-4-methylpiperidine-1-carboxylate in line with the synthesis described in 2.1 to 2.2 but using 2-azabicyclo[2.2.1]heptane hydrochloride instead of 4-fluoropiperidine hydrochloride in experimental procedure 2.1. LCMS [M−·Cl⁻]⁺ 294.4. ¹H NMR (Methanol-d₄, 400 MHz): δ (ppm) 4.32-4.09 (m, 4H), 3.59-3.23 (m, 7H), 3.21-2.97 (m, 4H), 2.88-2.72 (m, 1H), 2.34 (d, J=14.4 Hz, 2H), 2.05-1.97 (m, 1H), 1.86-1.68 (m, 5H), 1.62-1.49 (m, 1H), 1.31 (s, 3H).

### Example 5

### Synthesis of (S)—N-(2-hydroxy-3-(piperidin-1-yl)propoxy)-4-methylpiperidine-4-carbimidoyl chloride di-2,2-trifluoroacetate, NME200384

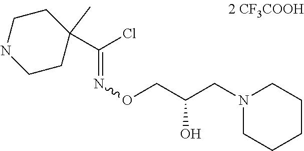 [SMILES: CC1(C(Cl)=NOC[C@@H](O)CN2CCCCC2)CCNCC1]

In a generally similar manner with non-critical variations was made (S)—N-(2-hydroxy-3-(piperidin-1-yl)propoxy)-4-methylpiperidine-4-carbimidoyl chloride di-2,2,2-trifluoroacetate (93 mg, 20.57%) as a yellow oil from the commercially available tert-butyl 4-(N′-hydroxycarbamimidoyl)-4-methylpiperidine-1-carboxylate in line with the synthesis described in 2.1 to 2.2 but using piperidine instead of 4-fluoropiperidine hydrochloride and using [(2S)-oxiran-2-yl]methyl 3-nitrobenzenesulfonate instead of oxiran-2-ylmethyl 3-nitrobenzenesulfonate in experimental procedure 4.1. LCMS [M−·Cl⁻]⁺ 282.0. ¹H NMR (Deuterium Oxide, 400 MHz): δ (ppm) 4.32-4.23 (m, 1H), 4.17-4.03 (m, 2H), 3.51-3.37 (m, 2H), 3.22-3.11 (m, 3H), 3.09-2.97 (m, 3H), 2.95-2.86 (m, 1H), 2.82 (t, J=12.7, 12.7 Hz, 1H), 2.23 (d, J=15.2 Hz, 2H), 1.86-1.74 (m, 2H), 1.74-1.55 (m, 5H), 1.43-1.28 (m, 1H), 1.18 (s, 3H)

### Example 6

### Synthesis of (R)—N-(2-hydroxy-3-(piperidin-1-yl)propoxy)-4-methylpiperidine-4-carbimidoyl chloride di-2,2,2-trifluoroacetate, NME200387

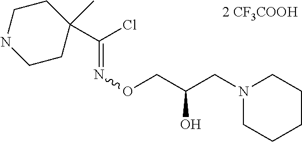 [SMILES: CC1(C(Cl)=NOC[C@H](O)CN2CCCCC2)CCNCC1]

In a generally similar manner with non-critical variations was made (R)—N-(2-hydroxy-3-(piperidin-1-yl)propoxy)-4-methylpiperidine-4-carbimidoyl chloride di-2,2,2-trifluoroacetate (450 mg, 82.13%) as a yellow oil from the commercially available tert-butyl 4-(N′-hydroxycarbamimidoyl)-4-methylpiperidine-1-carboxylate in line with the synthesis described in 2.1 to 2.2 but using piperidine instead of 4-fluoropiperidine hydrochloride and using [(2R)-oxiran-2-yl]methyl 3-nitrobenzenesulfonate instead of oxiran-2-ylmethyl 3-nitrobenzenesulfonate in experimental procedure 2.1. LCMS [M−·Cl⁻]⁺ 282.0. ¹H NMR (DMSO-d₆, 400 MHz): δ (ppm) 9.35 (s, 1H), 8.86 (s, 1H), 8.68 (s, 1H), 4.26-4.19 (m, 1H), 4.16-4.05 (m, 2H), 3.47-3.38 (m, 2H), 3.26-2.73 (m, 9H), 2.11 (d, J=15.0 Hz, 2H), 1.80-1.58 (m, 7H), 1.43-1.34 (m, 1H), 1.23 (s, 3H)

### Example 7

### Synthesis of N-(2-hydroxy-3-(4-methylpiperazin-1-yl)propoxy)-4-(pyridin-3-ylmethyl)piperidine-4-carbimidoyl chloride tetra-2,2,2-trifluoroacetate, NME200513

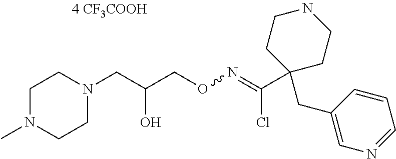 [SMILES: CN1CCN(CC(O)CON=C(Cl)C2(Cc3cccnc3)CCNCC2)CC1]

In a generally similar manner with non-critical variations was made N-(2-hydroxy-3-(4-methylpiperazin-1-yl)propoxy)-4-(pyridin-3-ylmethyl)piperidine-4-carbimidoyl chloride tetra-2,2,2-trifluoroacetate (344 mg, 23.14%) as a yellow oil from the commercially available tert-butyl 4-cyanopiperidine-1-carboxylate in line with the synthesis described in 1.4, 1.1, 2.1-2.2, but using 3-(bromomethyl)pyridine hydrobromide instead of 3-(bromomethyl)-5-fluoro-pyridine hydrobromide in experimental procedure 1.4 and using 1-methylpiperazine instead of 4-fluoropiperidine hydrochloride in experimental procedure 2.1. LCMS [M+1]⁺ 410.2. ¹H NMR (Deuterium Oxide, 400 MHz): δ (ppm) 8.57 (d, J=5.5 Hz, 1H), 8.45 (s, 1H), 8.23 (d, J=8.0 Hz, 1H), 7.87 (t, 1H), 4.23-4.13 (m, 1H), 3.99-3.93 (m, 1H), 3.92-3.87 (m, 1H), 3.83-3.57 (m, 4H), 3.54-3.29 (m, 4H), 3.26-3.18 (m, 4H), 3.08 (s, 2H), 2.95-2.86 (m, 5H), 2.22 (d, J=14.2 Hz, 2H), 1.81 (t, J=13.7, 13.7 Hz, 2H).

### Example 2: Determination of Potencies and Efficacies of Oximes Using GCase Assay

### Materials

Human fibroblast cell line GM10915 harboring the L444P GBA mutation was obtained from Coriell Biorepositories. All chemicals (Glacial acetic acid, Glycine, 4-Methylumbelliferyl b-D-glucopyranoside (4-MUG), Sodium acetate trihydrate, Sodium hydroxide, Crystal violet, SDS, Ammonium hydroxide) were obtained from Sigma-Aldrich (Denmark). Compounds tested for GCase activity were dissolved in H₂O or DMSO.

### Methods

The GM10915 cell line was cultured under standard cell culture conditions (37° C. and 5% CO₂) in complete DMEM medium supplemented with nonessential amino acids (NEAA), 1% Pen-Strep and 12% FCS. Cells were seeded at a density of 10⁴ cells/well in 100 μL complete medium in one black 96-well plate for glucosylceramidase (GCase) activity measurement and in one clear 96-well plate for crystal violet staining to correct for cell density. Crystal violet staining is performed to obtain quantitative information about the relative density of cells adhering to multi-wells plates.

### Assay of GCase Activity

The assay was adapted from Sawkar et al (2002) and briefly described in the following. The day after seeding of cells, the medium was replaced with fresh medium containing the compounds to be tested. Compounds were tested in duplicate and in an 8-point diluted dose range to obtain a dose response. Cells were exposed with compounds for five days. Fresh compound was added every 2-3 days. PBS was included to define the basal level of GCase activity.

Cells were washed three times with 200 μL PBS per well and 50 μL of 2.5 mM 4-MUG buffer (4-MUG dissolved in 0.2 M acetate buffer pH 4.0) was added and the cells were incubated at 37° C., 5% CO₂ for 23 hours. The reaction was stopped by adding 150 μL 0.2 M glycine buffer (pH 10.8). Fluorescence was measured with a Varioskan® Flash reader (Thermo Scientific) at an excitation/emission setting of 365/445 nm.

### Crystal Violet Staining

Cells were treated with compounds in a parallel setup identical to the setup to test for GCase activity. At the end of compound treatment, cells were washed once with 200 μL PBS per well and 50 μL 0.1% w/v crystal violet (in H₂O) was added. Following 10 min. of incubation, the crystal violet solution was removed, and the cells were washed three times with 200 μL PBS and 100 μL 1% SDS was added to solubilize the stain. The plate was agitated on an orbital shaker for 10-30 min. Absorbance (A) is measured at 570 nM using a Varioskan® Flash reader (Thermo Scientific).

### Calculations

The fluorescence signal (F) derived from the GCase measurement is normalized to the absorbance signal (A) derived from the crystal violet staining. The percent GCase activity resulting from compound treatment is calculated relative to the basal activity obtained from untreated cells.

$$Percent GCase activity = 100 * \frac{{( F / A )}_{compound}}{{( F / A )}_{untreated}}$$

The potency, EC_{1.5}, is determined based on the dose response effects of the compounds as the concentration where “Percent GCase activity”=150% corresponding to at 1.5-fold induction of GCase activity. Maximal effect of compounds (Emax) is determined from the dose response effects as the maximum “Percent GCase activity” achieved in the dose range tested.

### Results

The GBA potencies and Emax were determined as described above in the present example and the results are shown in Table 1 below.

| ID | Structure | GBA EC_{1.5} (μM) | GBA Emax (%) |
|---|---|---|---|
| Ambroxol reference | 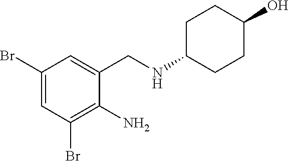 [SMILES: Nc1c(Br)cc(Br)cc1CN[C@H]1CC[C@H](O)CC1] | >20* *The 1.5-fold induction criteria was not reached within the tested concentration range | 127 |
| LTI-291 reference | 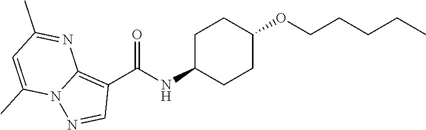 [SMILES: CCCCCO[C@H]1CC[C@H](NC(=O)c2cnn3c(C)cc(C)nc23)CC1] | 112 | 232 |
| 524 (II) | 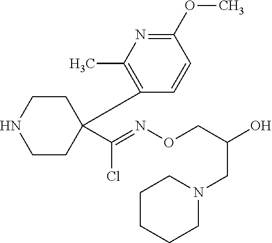 [SMILES: COc1ccc(CC2(/C(Cl)=N/OCC(O)CN3CCCCC3)CCNCC2)c(C)n1] | 1 | 149 |
| 525 (II) | 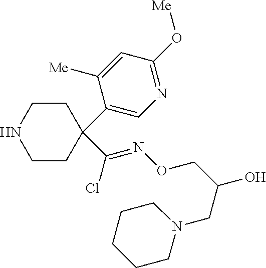 [SMILES: COc1cc(C)c(C2(/C(Cl)=N/OCC(O)CN3CCCCC3)CCNCC2)cn1] | 3 | 168 |
| 563 (I) | 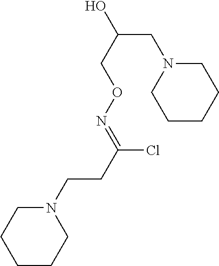 [SMILES: OC(CO/N=C(\Cl)CCN1CCCCC1)CN1CCCCC1] | 3 | 211 |
| 562 (I) | 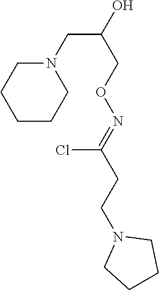 [SMILES: OC(CO/N=C(\Cl)CCN1CCCC1)CN1CCCCC1] | 3 | 239 |
| 446 (I) | 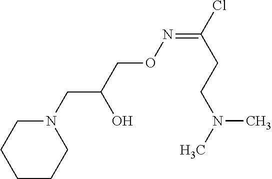 [SMILES: CN(C)CC/C(Cl)=N\OCC(O)CN1CCCCC1] | 6 | 176 |
| 534 (II) | 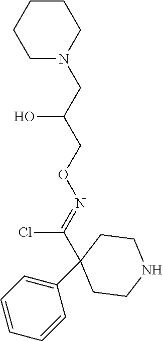 [SMILES: OC(CO/N=C(\Cl)C1(c2ccccc2)CCNCC1)CN1CCCCC1] | 19 | 226 |
| 504 (III) | 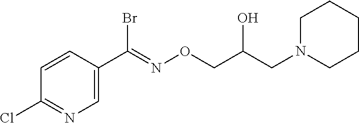 [SMILES: OC(CO/N=C(\Br)c1ccc(Cl)nc1)CN1CCCCC1] | 20 | 310 |
| 419 (II) | 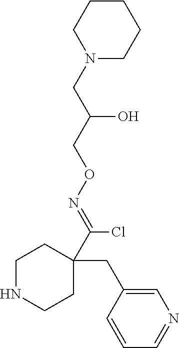 [SMILES: OC(CO/N=C(\Cl)C1(Cc2cccnc2)CCNCC1)CN1CCCCC1] | 12 | 387 |
| 452 (III) | 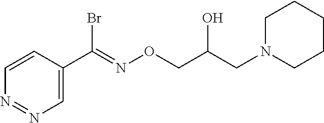 [SMILES: OC(CO/N=C(\Br)c1ccnnc1)CN1CCCCC1] | 18 | 572 |
| 466 (III) | 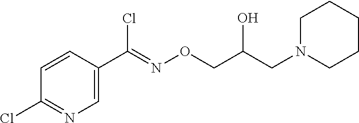 [SMILES: OC(CO/N=C(\Cl)c1ccc(Cl)nc1)CN1CCCCC1] | 18 | 221 |
| 493 (II) | 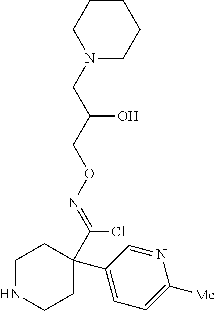 [SMILES: Cc1ccc(C2(/C(Cl)=N/OCC(O)CN3CCCCC3)CCNCC2)cn1] | 18 | 279 |
| 501 (III) | 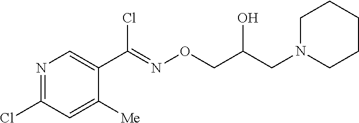 [SMILES: Cc1cc(Cl)ncc1/C(Cl)=N/OCC(O)CN1CCCCC1] | 19 | 128 |
| 415 (II) | 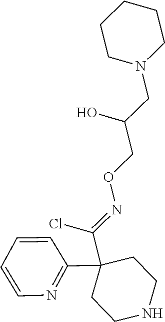 [SMILES: OC(CO/N=C(\Cl)C1(c2ccccn2)CCNCC1)CN1CCCCC1] | 23 | 235 |
| 502 (III) | 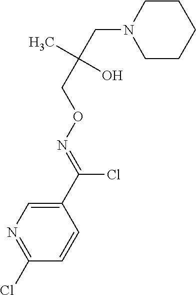 [SMILES: CC(O)(CO/N=C(\Cl)c1ccc(Cl)nc1)CN1CCCCC1] | 23 | 131 |
| 475 (III) | 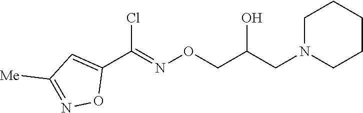 [SMILES: Cc1cc(/C(Cl)=N/OCC(O)CN2CCCCC2)on1] | 24 | 354 |
| 409 (II) | 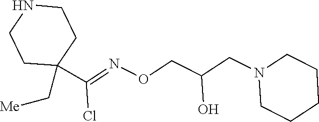 [SMILES: CCC1(/C(Cl)=N/OCC(O)CN2CCCCC2)CCNCC1] | 25 | 255 |
| 550 (II) | 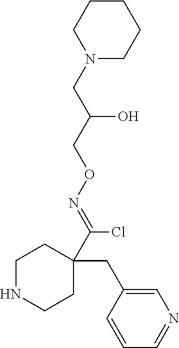 [SMILES: OC(CO/N=C(\Cl)C1(Cc2cccnc2)CCNCC1)CN1CCCCC1] | 27 | 159 |
| 532 (I) | 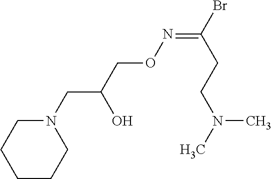 [SMILES: CN(C)CC/C(Br)=N\OCC(O)CN1CCCCC1] | 30 | 239 |
| 384 (II) | 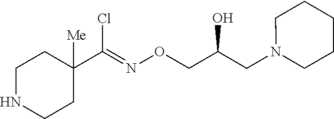 [SMILES: CC1(/C(Cl)=N/OC[C@@H](O)CN2CCCCC2)CCNCC1] | 32 | 352 |
| 448 (II) | 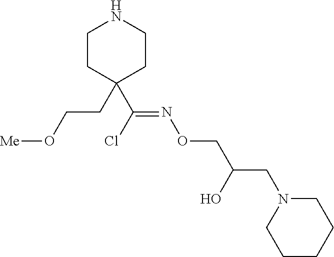 [SMILES: COCCC1(/C(Cl)=N/OCC(O)CN2CCCCC2)CCNCC1] | 34 | 468 |
| 399 (II) | 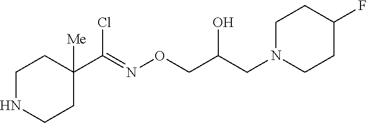 [SMILES: CC1(/C(Cl)=N/OCC(O)CN2CCC(F)CC2)CCNCC1] | 34 | 314 |
| 257 (III) | 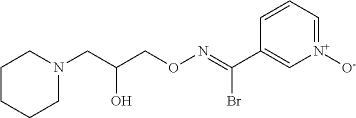 [SMILES: [O-][n+]1cccc(/C(Br)=N/OCC(O)CN2CCCCC2)c1] | 35 | 307 |
| 387 (II) | 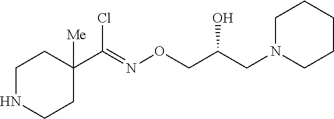 [SMILES: CC1(/C(Cl)=N/OC[C@H](O)CN2CCCCC2)CCNCC1] | 35 | 379 |
| 535 (II) | 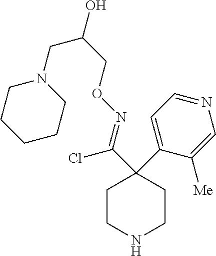 [SMILES: Cc1cnccc1C1(/C(Cl)=N/OCC(O)CN2CCCCC2)CCNCC1] | 36 | 186 |
| 336 (II) | 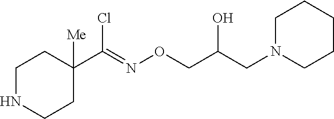 [SMILES: CC1(/C(Cl)=N/OCC(O)CN2CCCCC2)CCNCC1] | 37 | 320 |
| 453 (III) | 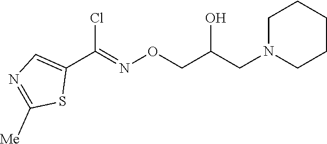 [SMILES: Cc1ncc(/C(Cl)=N/OCC(O)CN2CCCCC2)s1] | 39 | 202 |
| 492 (II) | 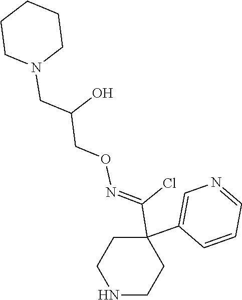 [SMILES: OC(CO/N=C(\Cl)C1(c2cccnc2)CCNCC1)CN1CCCCC1] | 42 | 383 |
| 410 (II) | 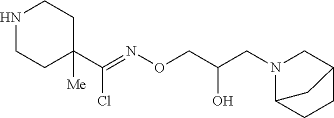 [SMILES: CC1(/C(Cl)=N/OCC(O)CN2CC3CCC2C3)CCNCC1] | 43 | 344 |
| 352 (III) | 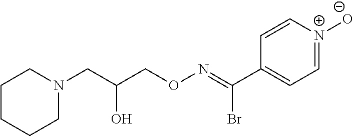 [SMILES: [O-][n+]1ccc(/C(Br)=N/OCC(O)CN2CCCCC2)cc1] | 45 | 474 |
| 513 (II) | 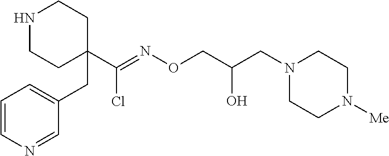 [SMILES: CN1CCN(CC(O)CO/N=C(\Cl)C2(Cc3cccnc3)CCNCC2)CC1] | 35 | 174 |
| 403 (II) | 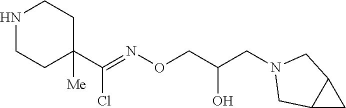 [SMILES: CC1(/C(Cl)=N/OCC(O)CN2CC3CC3C2)CCNCC1] | 48 | 264 |
| 402 (II) | 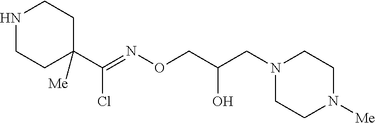 [SMILES: CN1CCN(CC(O)CO/N=C(\Cl)C2(C)CCNCC2)CC1] | 50 | 408 |
| 503 (III) | 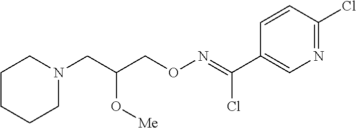 [SMILES: COC(CO/N=C(\Cl)c1ccc(Cl)nc1)CN1CCCCC1] | 50 | 142 |
| 512 (II) | 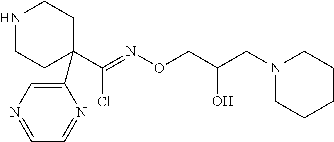 [SMILES: OC(CO/N=C(\Cl)C1(c2cnccn2)CCNCC1)CN1CCCCC1] | 50 | 147 |
| 593 (I) | 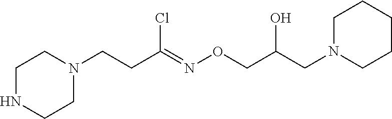 [SMILES: OC(CO/N=C(\Cl)CCN1CCNCC1)CN1CCCCC1] | 50 | 136 |
| 599 (I) | 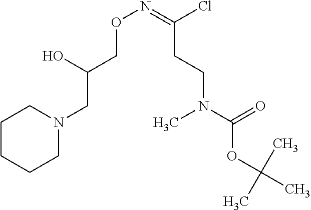 [SMILES: CN(CC/C(Cl)=N\OCC(O)CN1CCCCC1)C(=O)OC(C)(C)C] | 48 | 136 |

### CONCLUSION

This example demonstrates that the oximes of the present disclosure are highly potent and efficacious in comparison with state-of-the-art GBA inducers like Ambroxol and LTI-291. These effects render the oximes of the present disclosure promising candidates for treatment of GBA-mediated disorders.

1. A compound of formula (I), 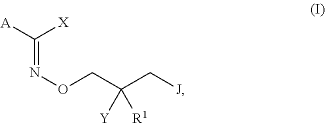
or a pharmaceutically acceptable salt thereof, wherein
R¹ is selected from the group consisting of: H, C₁₋₆ alkyl, and halogen;
Y is selected from the group consisting of: OH, and C₁₋₆ alkoxy;
X is selected from the group consisting of: chlorine and bromine;
J is an aliphatic cycle comprising at least one nitrogen atom, wherein the aliphatic cycle is optionally substituted; and wherein
A is selected from the group consisting of: 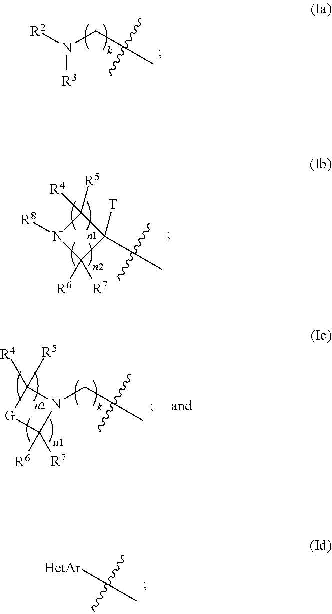
wherein each k is 1, 2, 3, or 4;
R² and R³ are independently of each other selected from the group consisting of: C₁₋₆ alkyl, C₁₋₆ acyl, and CO₂—C₁₋₆ alkyl;
n1, n2, u1, and u2 are independently of each other selected from the group consisting of: 1, 2, or 3;
each R⁴ is independently selected from the group consisting of: H, halogen, and C₁₋₄ alkyl, wherein each methylene group optionally is replaced by —O—;
each R⁵ is independently selected from the group consisting of: H, halogen, and C₁₋₄ alkyl, wherein each methylene group optionally is replaced by —O—;
each R⁶ is independently selected from the group consisting of: H, halogen, and C₁₋₄ alkyl, wherein each methylene group optionally is replaced by —O—;
each R⁷ is independently selected from the group consisting of: H, halogen, and C₁₋₄ alkyl, wherein each methylene group optionally is replaced by —O—;
R⁸ is selected from the group consisting of: H, and C₁₋₄ alkyl;
G is selected from the group consisting of: —CH₂—, —CH(R⁹)—, —C(R⁹)₂—, —NH—, and —N(R⁹)—;
wherein each R⁹ is independently selected from the group consisting of hydrogen, halogen, C₁₋₄ alkyl, wherein each methylene group optionally is replaced by —O—; and wherein
HetAr is a heteroaryl optionally substituted by one or more R¹⁰, wherein each R¹⁰ is independently selected from the group consisting of: hydrogen, C₁₋₆ alkyl, halogen, hydroxy, C₁₋₆ alkoxy, amino, amido, and C₁₋₆ acyl;
and wherein T is selected from the group consisting of: 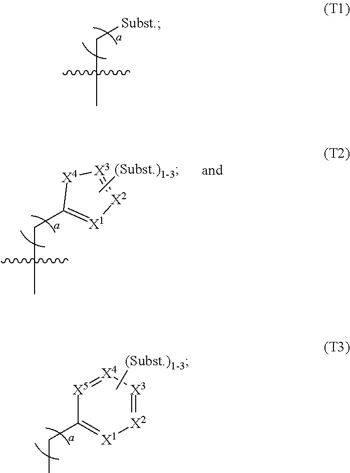
or any tautomer thereof,
wherein a is 0, 1, 2, or 3;
X¹, X², X³, X⁴, and X⁵ independently are selected from the group consisting of: C, CH, and N; and
each one, two, or three Subst. is independently selected from the group consisting of: hydrogen, alkyl, halogen, hydroxy, alkoxy, amino, amido, acyl, cycloalkyl, and heterocycloalkyl, wherein each methylene group of the alkyl is optionally replaced by —O—;
with the proviso that when X is chlorine, A is not

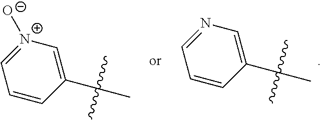 [SMILES: *c1ccc[n+]([O-])c1 ; *c1cccnc1]

2. The compound according to claim 1, wherein J is of formula (J1): 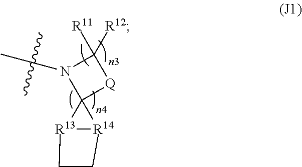
wherein n3 and n4 are independently 1, 2, or 3;
each R¹¹ is independently selected from the group consisting of: H, halogen, and C₁₋₄ alkyl, wherein each methylene group optionally is replaced by —O—;
each R¹² is independently selected from the group consisting of: H, halogen, and C₁₋₄ alkyl, wherein each methylene group optionally is replaced by —O—;
each R¹³ is independently selected from the group consisting of: H, halogen, and C₁₋₄ alkyl, wherein each methylene group optionally is replaced by —O—;
each R¹⁴ is independently selected from the group consisting of: H, halogen, and C₁₋₄ alkyl, wherein each methylene group optionally is replaced by —O—;
Q is selected from the group consisting of: a bond, —CH₂—, —CH(R¹⁵)—, —C(R¹⁵)₂—, —NH—, and —N(R¹⁵)—; and wherein each R¹⁵ is independently selected from the group consisting of hydrogen, halogen, C₁₋₄ alkyl, wherein each methylene group optionally is replaced by —O—; and wherein a pair of R¹¹ and R¹³ are optionally linked together to form a ring.

3. The compound according to claim 2, wherein Q is selected from the group consisting of: a bond, —CH₂—, —CHF—, —N(Me)-, and —NH—.

4. The compound according to any one of the preceding claims, wherein J is selected from the group consisting of:

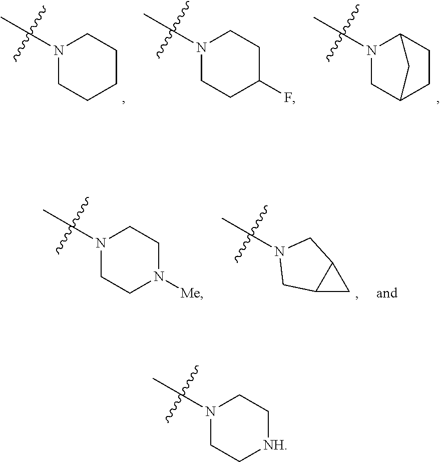 [SMILES: *N1CCCCC1 ; *N1CCC(F)CC1 ; *N1CC2CCC1C2 ; *N1CCN(C)CC1 ; *N1CC2CC2C1 ; *N1CCNCC1]

5. The compound according to any one of the preceding claims, wherein Y is OH.

6. The compound according to any one of the preceding claims, wherein A is of formula (Ia); 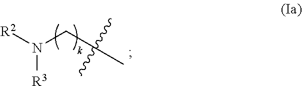
k is 2, R³ is C₁₋₃ alkyl, and R² is C₁₋₃ alkyl or CO₂tBu.

7. The compound according to any one of the preceding claims, wherein A is of formula (Ib); 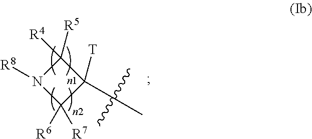
wherein n1 and n2 are each 2; R⁴, R⁵, R⁶, and R⁷ are each hydrogen, R⁸ is hydrogen or C₁₋₃ alkyl; and T is of formula (T3); 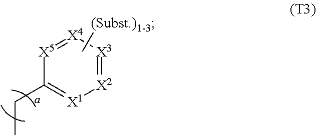
wherein a is 0 or 1; wherein X¹, X², and/or X³ is N and the remainder of X¹—X⁵ are independently C or CH; and wherein each one, two, or three Subst. is independently selected from the group consisting of: hydrogen, C₁₋₄ alkyl, halogen, hydroxy, C₁₋₄ alkoxy, and C₁₋₄ acyl.

8. The compound according to any one of the preceding claims, wherein only one Subst. is present and is:
a) methyl; or
b) chlorine.

9. The compound according to any one claims 1-5, wherein A is of formula (Ib); 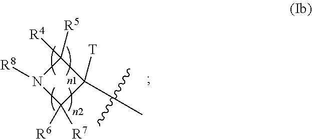
wherein n1 and n2 are each 2; R⁴, R⁵, R⁶, and R⁷ are each hydrogen; R⁸ is hydrogen or C₁₋₃ alkyl; and T is of formula (T1); 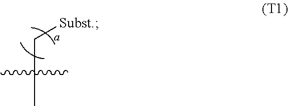
wherein a is 0 and Subst is C₁₋₆ alkyl, wherein each methylene group is optionally replaced by —O—.

10. The compound according to any one claims 1-5, wherein A is of formula (Ic); 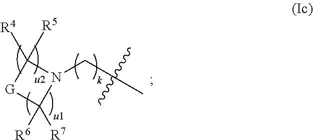
wherein k is 1, 2, or 3; u1 and u2 are each 1 or 2; R⁴, R⁵, R⁶, and R⁷ are each hydrogen; and G is selected from the group consisting of: a bond, —CH₂—, —NH—, and —N(C₁₋₃ alkyl)-.

11. The compound according to any one of claims 1-5, wherein A is of formula (Id); 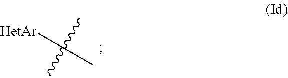
wherein HetAr is a C₅₋₁₃ heteroaryl comprising one or more ring heteroatoms selected from nitrogen, oxygen and sulfur, and which is monocyclic, bicyclic, or tricyclic.

12. The compound according to any one of the preceding claims, wherein A is selected from the group consisting of:

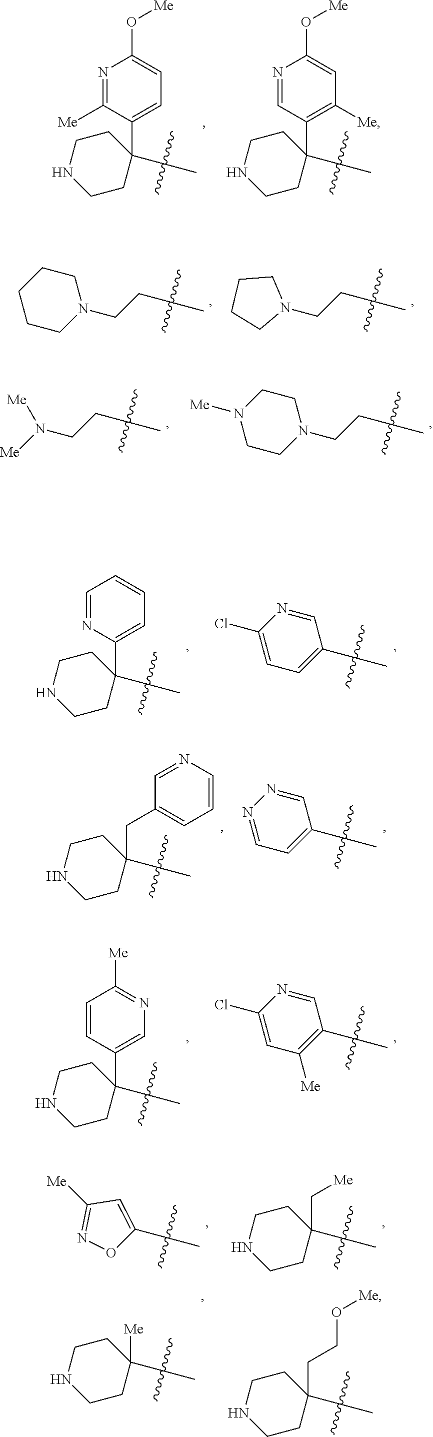

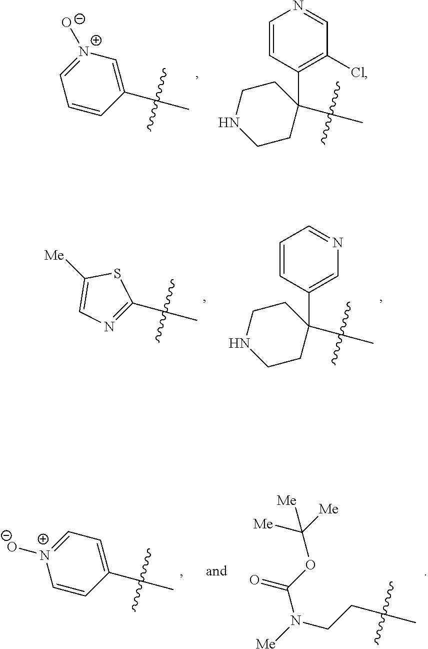 [SMILES: *c1ccc[n+]([O-])c1 ; *C1(c2ccncc2Cl)CCNCC1 ; *c1ncc(C)s1 ; *C1(c2cccnc2)CCNCC1 ; *c1cc[n+]([O-])cc1 ; *CCN(C)C(=O)OC(C)(C)C]

13. The compound according to claim 1, wherein the compound is selected from the group consisting of:

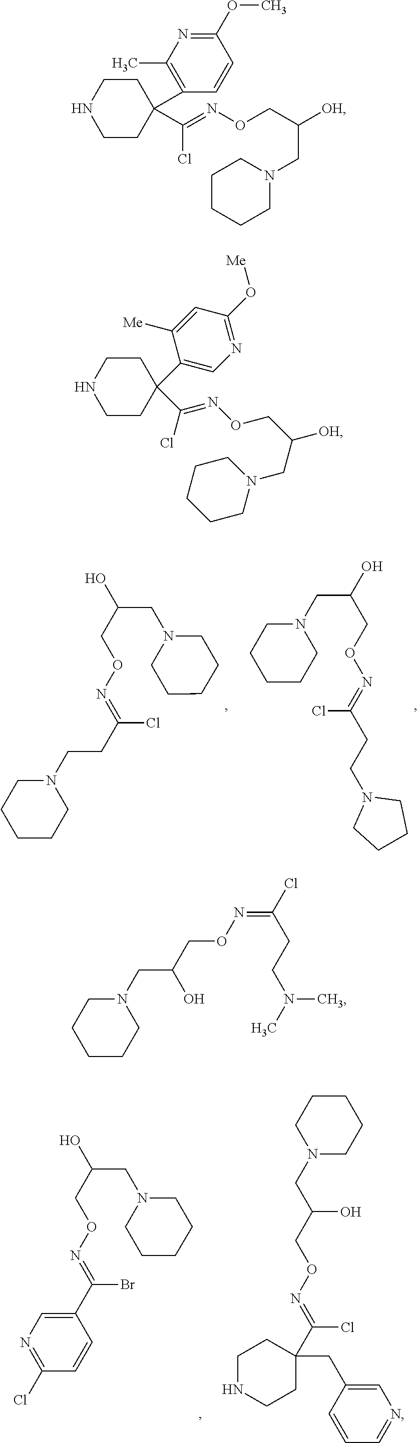

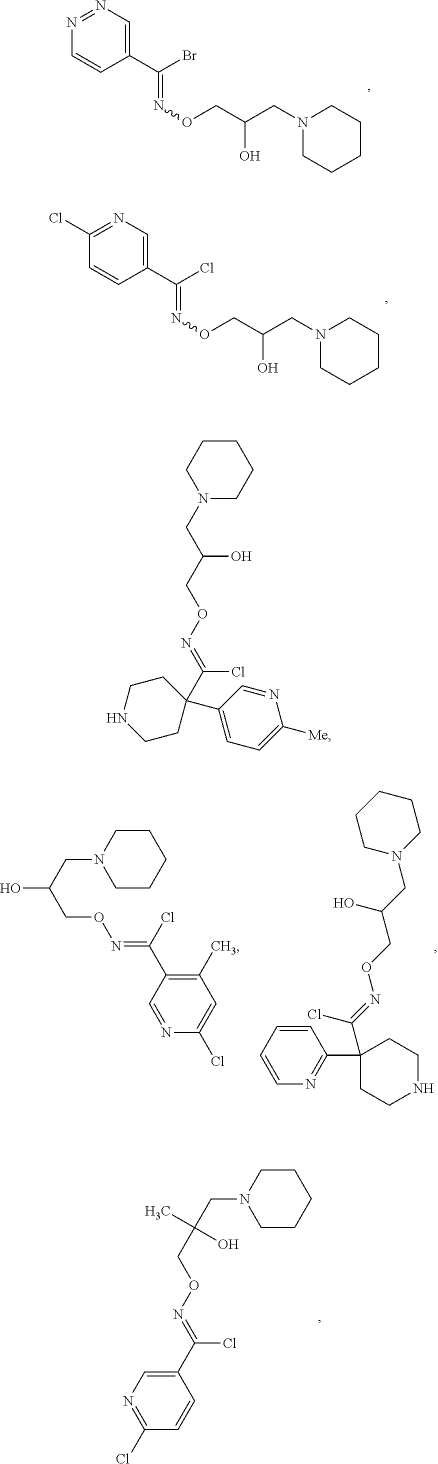

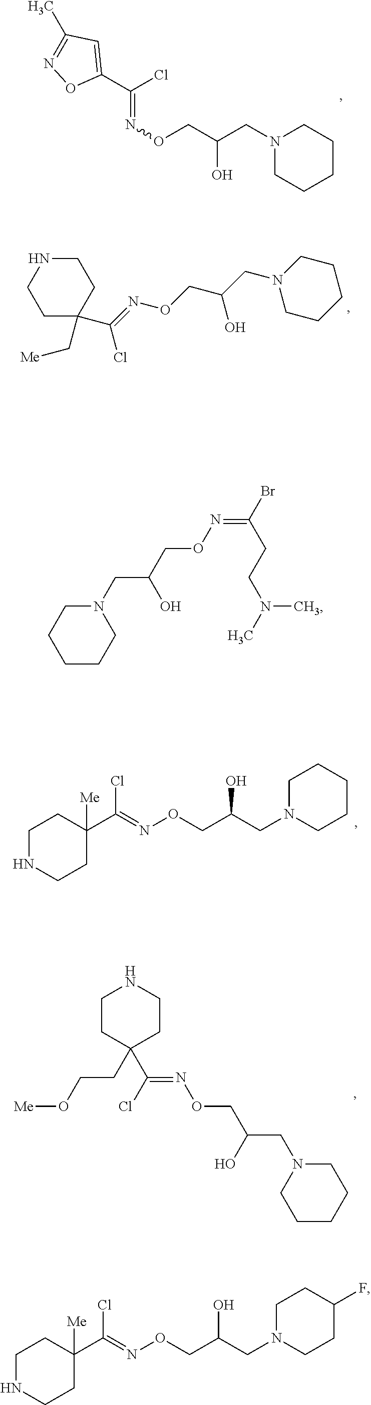 [SMILES: Cc1cc(C(Cl)=NOCC(O)CN2CCCCC2)on1 ; CCC1(/C(Cl)=N/OCC(O)CN2CCCCC2)CCNCC1 ; CN(C)CC/C(Br)=N\OCC(O)CN1CCCCC1 ; CC1(/C(Cl)=N/OC[C@@H](O)CN2CCCCC2)CCNCC1 ; COCCC1(/C(Cl)=N/OCC(O)CN2CCCCC2)CCNCC1 ; CC1(/C(Cl)=N/OCC(O)CN2CCC(F)CC2)CCNCC1]

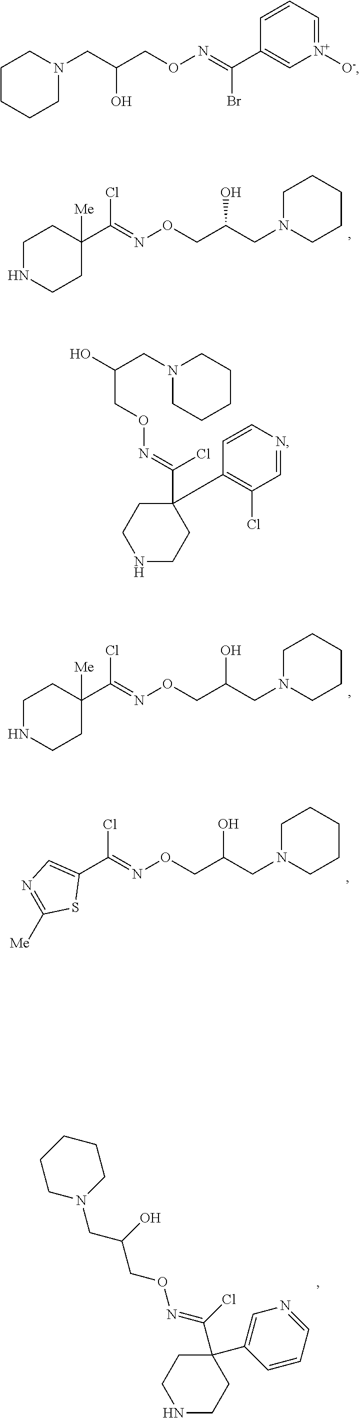

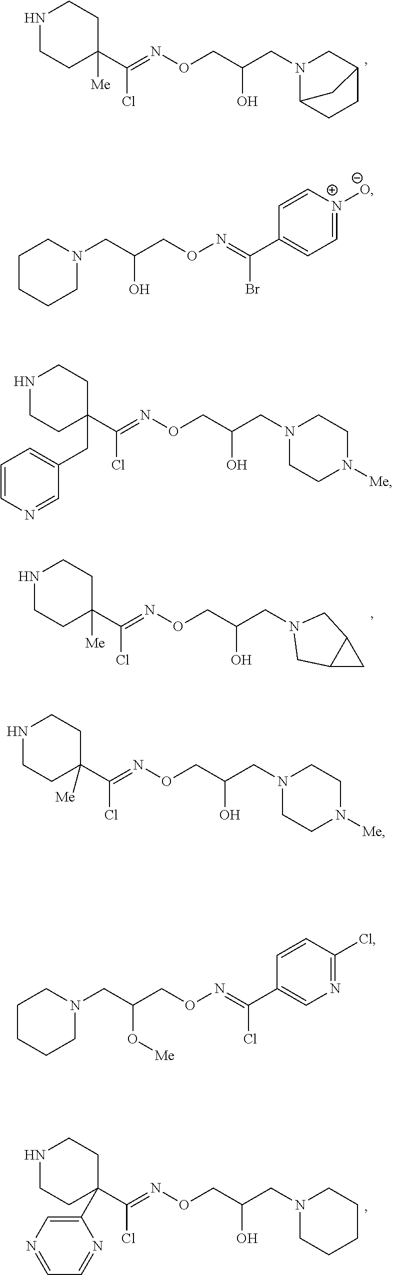

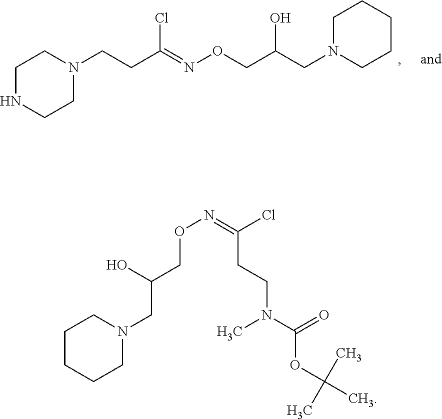 [SMILES: OC(CO/N=C(\Cl)CCN1CCNCC1)CN1CCCCC1 ; CN(CC/C(Cl)=N\OCC(O)CN1CCCCC1)C(=O)OC(C)(C)C]

14. The compound according to any one of the preceding claims, wherein the compound is a GBA inducer and increases glucocerebrosidase (GBA) enzyme levels and/or GBA enzyme activity.

15. A compound as defined in any one of claims 1-14 for use in the treatment of Parkinson's disease (PD).
